(19) United States
(12) Patent Application Publication
(10) Pub. No.: US 2009/0017004 A1
(43) Pub. Date: Jan. 15, 2009

(54) POLYMERIC DRUG DELIVERY SYSTEMS CONTAINING AN AROMATIC ALLYLIC ACID

(75) Inventor: Hong ZHAO, Edison, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS, INC., Bridgewater, NJ (US)

(21) Appl. No.: 12/171,769

(22) Filed: Jul. 11, 2008

Related U.S. Application Data
(60) Provisional application No. 60/949,195, filed on Jul. 11, 2007.

(51) Int. Cl. A61K 38/50; C07D 207/46; C07D 207/12; A61P 43/00; C07D 207/24; C08F 122/40

(52) U.S. Cl. 424/94.6; 548/542; 548/546; 526/262

(57) ABSTRACT

The present invention provides polymeric delivery systems including an aromatic allyllic acyl group. Methods of making the polymeric delivery systems and methods of treating mammals using the same are also disclosed.

### CROSS-REFERENCE TO RELATED APPLICATION

This application claims the benefit of priority from U.S. Provisional Patent Application Ser. No. 60/949,195 filed Jul. 11, 2007, the contents of which are incorporated herein by reference.

### FIELD OF THE INVENTION

The present invention relates to drug delivery systems. In particular, the invention relates to polymeric-based drug delivery systems including an aromatic allylic acyl moiety.

### BACKGROUND OF THE INVENTION

Medicinal agents can be relatively easily included in pharmaceutical formulations when they are available as water-soluble salt forms. Problems arise, however, when a desired medicinal agent is insoluble in aqueous fluid or rapidly degraded in vivo. For example, it is often difficult to solubilize medicinal agents such as alkaloids, and proteins are often prematurely degraded upon administration into the body.

Over the years, various proposals have been made to resolve the obstacles. One of the attempts to solve the obstacles is to include such medicinal agents as part of a soluble transport system. Such transport systems can include permanent conjugate-based systems or prodrugs. In particular, polymeric transport systems can improve the solubility and stability of medicinal agents.

Multifunctional therapeutics such as proteins can be employed in permanent conjugate-based transport systems including polymers. Proteins employed in such systems maintain biological activities to achieve therapeutic effects. Examples of polymeric conjugates of proteins are described in U.S. Pat. No. 4,179,337, the disclosure of which is incorporated herein by reference.

On the other hand, prodrugs are often biologically inert or substantially inactive forms of a parent or active drug. Among many factors which influence the rate of release of the parent drug, i.e. the rate of hydrolysis, the release rate is especially modified by the linkages joining the parent drug to the rest of the prodrug system. Care must thus be taken to avoid the prodrugs from being eliminated through the kidney or reticular endothelial system, etc. before a sufficient amount of hydrolysis occurs to release the parent drug. Prodrugs including polymers can improve the circulating half-life of the drug. The prodrug linkages can modify in vivo hydrolysis to a rate which eventually generates sufficient amounts of the parent drug after administration thereby providing improved control of the pharmacokinetics of therapeutic moieties like small molecule drugs and the like. Some examples of polymeric prodrugs are described in commonly-assigned U.S. Pat. Nos. 6,180,095 and 6,720,306, the contents of each of which are incorporated herein by reference.

In spite of the attempts and advances, there continues to be a need to improve polymeric delivery platforms. The present invention addresses this need and others.

### SUMMARY OF THE INVENTION

In one aspect of the invention, there are provided compounds of Formula (I).

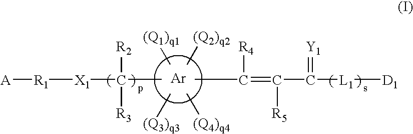

wherein:

A is a capping group or

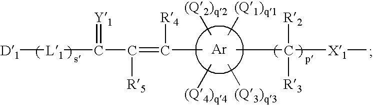

R₁ is a substantially non-antigenic water-soluble polymer;

X₁ and X′₁ are independently O, S, SO, SO₂, NR₆ or a bond;

Ar and Ar′ are independently an aryl or heteroaryl moiety;

Y₁ and Y′₁ are independently O, S, or NR₆;

L₁ and L′₁ are independently selected bifunctional linkers;

D₁ and D′₁ are independently selected from among hydrogen, OH, leaving groups, functional groups, targeting groups, diagnostic agents and biologically active moieties;

(p) and (p′) are independently 0 or a positive integer, preferably zero or one;

(q₁), (q′₁), (q₂) (q′₂), (q₃), (q′₃), (q₄) and (q′₄) are independently zero or one;

(s) and (s′) are independently zero or a positive integer:

R₂, R′₂, R₃, R′₃ and R₆, are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy, and

R₄, R′₄, R₅, R′₅, Q₁₋₄ and Q′₁₋₄ are independently selected from among the same moieties which can be used for R₂ or each can be:

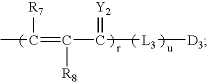

wherein
R₇ and R₈ are independently selected from the same group as that which defines R₂;

Y₂ is O, S or NR₆;

L₃ is a bifunctional linker;

(r) is independently zero or one;

(u) is zero or a positive integer; and

D₃ is selected from among hydrogen, OH, leaving groups, functional groups, targeting groups and biologically active moieties; and

provided that (r) is not zero when (u) is zero.

In certain preferred aspects of the invention, the polymeric drug-delivery systems include cinnamic acid.

In some preferred aspects, at least one functional group attached to the aromatic portion of the systems is conjugated to a targeting moiety.

In some particularly preferred aspects, R₁ includes a linear, branched or multi-armed poly(ethylene glycol) residue with molecular weight of from about 5,000 to about 60,000; and (p) is zero or one; and Y₁ is O. In one particular aspect, R₂₋₅ and R′₂₋₅ are selected from among hydrogen, methyl and ethyl, and each is more preferably hydrogen.

In another aspect of the invention, there are provided methods of preparing the compounds described herein and methods of treatment using the compounds described herein.

One advantage of the aromatic allylic acid-based polymeric transport systems described herein is that the artisans are able to increase the loadings of medicinal agents.

Another advantage of the aromatic moiety-based polymeric transport systems described herein is that the polymeric delivery systems have improved stability. Without being bound by any theories, the hydrophobic microenvironment around the covalent linkage between polymers and a moiety such functional groups, biologically active moieties and targeting groups, protects the covalent linkage from exposure to basic aqueous medium or enzymes which can modify the covalent linkage, thereby stabilizing the covalent linkage. The stability of the polymeric systems also allows long-term storage prior to attaching to targeting groups or biologically active moieties.

A further advantage of the polymeric systems described herein allows attaching a second agent. Substitution can be easily arranged on the aromatic ring so that artisans in the art can attach a second drug to have synergistic effect for therapy or a targeting group for selectively targeted delivery. The polymeric delivery systems described herein allow targeting medicinal agents into the site of treatment. The aromatic moieties and double bond of the polymeric systems can be substituted with targeting moieties via chemical functional moieties.

The polymeric delivery systems containing an aromatic allylic acyl group can also improve the conjugation efficiency of therapeutic agents and thereby reduce the cost of manufacturing. Yet another advantage of the present invention allows preparing the polymeric systems herein in high purity and thereby having uniform pharmacokinetic properties.

Yet another advantage is that multiple steps previously required to attach a second agent can be avoided. For example, certain bifunctional groups can be directly attached to a second agent and therefore eliminate steps for activating the polymeric systems.

For purposes of the present invention, the term “residue” shall be understood to mean that portion of a compound, to which it refers, i.e. PEG, etc. that remains after it has undergone a substitution reaction with another compound.

For purposes of the present invention, the terms “a biologically active moiety” and “a residue of a biologically active moiety” shall be understood to mean that portion of a biologically active compound which remains after the biologically active compound has undergone a substitution reaction in which the transport carrier portion has been attached.

For purposes of the present invention, the term “polymeric residue” or “PEG residue” shall each be understood to mean that portion of the polymer or PEG which remains after it has undergone a reaction with other compounds, moieties, etc.

For purposes of the present invention, the term “alkyl” as used herein refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, C₁₋₆ hydrocarbonyl, groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” as used herein refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing, at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alkyl group substituted with a C₃₋₈ cycloalkyl group. Examples of cycloalkylalkyl groups include cyclopropylmethyl and cyclopentylethyl.

For purposes of the present invention, the term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “alkyl-thio-alkyl” refers to an alkyl-S-alkyl thioether, for example, methylthiomethyl or methylthioethyl.

For purposes of the present invention, the term “amino” as used herein refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

For purposes of the present invention, the term “alkylcarbonyl” refers to a carbonyl group substituted with alkyl group.

For purposes of the present invention, the terms “halogen” or “halo” as used herein refer to fluorine, chlorine, bromine, and iodine.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, the term “heteroatom” refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill, i.e., preferably from 1 to about 10, more preferably 1 or 2 in some embodiments.

For purposes of the present invention, the term “linked” shall be understood to include covalent (preferably) or noncovalent attachment of one group to another, i.e., as a result of a chemical reaction.

For purposes of the present invention, the term “bond” shall be understood to mean that an atom is absent and moieties adjacent to the group designated as “bond” are linked directly.

For purposes of the present invention, it shall be understood to mean that the pharmaceutically active compounds include small molecular weight molecules. Typically, the pharmaceutically active compounds have a molecular weight of less than about 1,500 daltons and optionally derivatized with amine-, hydroxyl- or thiol-containing moieties to provide a reactive site for conjugation with polymer.

The terms “effective amounts” and “sufficient amounts” for purposes of the present invention shall mean an amount which achieves a desired effect or therapeutic effect as such effect is understood by those of ordinary skill in the art.

Broadly speaking, successful treatment (i.e. tumor growth inhibition or inhibition of inflammation) shall be deemed to occur when the desired response is obtained, compared to that observed in the absence of the treatment with the compound described herein. For example, successful treatment, (i.e., tumor growth inhibition or inhibition of inflammation) can be defined by obtaining e.g., 10% or higher (i.e. 20% 30%, 40%) down regulation or up-regulation of genes associated with cancer or inflammation.

Furthers the use of singular terms for convenience in description is in no way intended to be so limiting. Thus, for example, reference to a composition comprising an enzyme refers to one or more molecules of that enzyme. It is also to be understood that this invention is not limited to the particular configurations, process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited by the appended claims and equivalents thereof

### BRIEF DESCRIPTION OF THE DRAWINGS

FIG. 1 schematically illustrates methods of synthesis described in Examples 1-7.

FIG. 2 schematically illustrates methods of synthesis described in Example 8.

### DETAILED DESCRIPTION OF THE INVENTION

### A. Overview

In one aspect of the present invention, there are provided compounds of Formula (I):

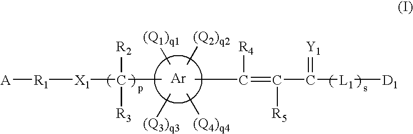

wherein:

A is a capping group or

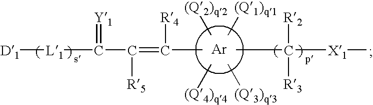

R₁ is a substantially non-antigenic water-soluble polymer;

X₁ and X′₁ are independently O, S, SO, SO₂, NR₆ or a bond;

Ar and Ar′ are independently an aryl or heteroaryl moiety;

Y₁ and Y′₁ are independently O, S, or NR₆, and preferably Y₁ and Y′₁ are O;

L₁ and L′₁ are independently selected bifunctional linkers;

D₁ and D′₁ are independently selected from among hydrogen, OH, leaving groups, functional groups, targeting groups, diagnostic agents and biologically active moieties including pharmaceutically active, small molecular weight compounds;

(p) and (p′) are independently zero or a positive integer, preferably from about 0 to about 10 (e.g., 0, 1, 2, 3, 4, 5, 6, 7, 8, 9 or 10), more preferably from about 0 to about 6 (e.g., 0, 1, 2, 3, 4, 5 or 6), and most preferably 0, 1 or 2;

(q₁), (q′₁), (q₂), (q′₂), (q₃), (q′₃), (q₄) and (q′₄) are independently zero or one;

(s) and (s′) are independently zero or a positive integer, preferably from about 0 to about 6 (e.g., 0, 1, 2, 3, 4, 5 or 6) and more preferably 0, 1, or 2;

R₂, R′₂, R₃, R′₃, and R₆ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆, alkoxycarbonyl, aryloxycarbonyl C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy, and

R₄, R′₄, R₅, R′₅, Q₁₋₄ and Q′₁₋₄ are independently selected from the same moieties which can be used for R₂ or each can be

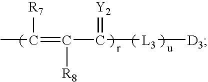

wherein
R₇ and R₈ are independently selected from the same group as that which defines R₂;
Y₂ is O, S or NR₆;
L₃ is a bifunctional linker;
(r) is zero or one;
(u) is zero or a positive integer, preferably from about 0 to about 4 (e.g., 0, 1, 2, 3 4, 5 or 6), more preferably zero, 1 or 2, and yet more preferably 3; and
D₃ is selected from among hydrogen, OH, leaving groups, functional groups, targeting groups and biologically active moieties; and
   provided that (r) is not zero when (u) is zero.

In one preferred aspect, X₁ and X′₁ are independently O, S, SO or SO₂ when (p) is zero.

In alternative aspects, the compounds described herein are selected such that the sum of (q₁)+(q₂)+(q₃)+(q₄) is not zero (e.g., preferably 1), and at least one (e.g., one, two or three) of R₄, R′₄, R₅, R′₅, Q₁₋₄ and Q′₁₋₄ is

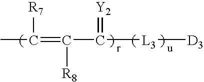

wherein D₃ is selected from the group consisting of leaving groups, functional groups, targeting groups, diagnostic agents and biologically active moieties. With this aspect, the leaving group is preferably selected from among N-hydroxysuccinimidyl, para-nitrophenoxy, ortho-nitrophenoxy and C₁-C₆ alkyloxy, and the functional group is preferably selected from among maleimidyl, vinyl, and residues of sulfone.

Within those aspects of the invention, the substituents contemplated for substitution, where said moieties corresponding to R₂, R′₂, R₃, R′₃, R₆, R₇, and R₈ are indicated as being possibly substituted can include, for example, acyl, amino, amido, amidine, ara-alkyl, aryl, azido, alkylmercapto, arylmercapto, carbonyl, carboxylate, cyano, ester, ether, formyl, halogen, heteroaryl, heterocycloalkyl, hydroxy, imino, nitro, thiocarbonyl, thioester-, thioacetate, thioformate, alkoxy, phosphoryl, phosphonate, phosphinate, silyl, sulfhydryl, sulfate, sulfonate, sulfamoyl, sulfonamide, and sulfonyl.

For purposes of the present invention, C(R₂)(R₃) is the same or different when (p) and/or (p′) are equal to or greater than 2.

For purposes of the present invention, each of L₁. L′₁ and L₃ are the same or different when (s), (s′) and (u) are equal to or greater than 2.

In certain aspects of the invention, when (p) is zero, there are preferably a sufficient number atoms, e.g., more than five or six atoms, present between X₁/X′₁ and C(═Y₁)/C(═Y′₁), so that a releasable cyclic moiety is not formed. For example, when (p) is zero, the polymeric portion attached to the phenyl ring would not be in an ortho position in relation to D₁, D′₁ or D₃.

In another aspect of the invention, the biological moieties include amine containing moieties, hydroxyl containing moieties and thiol containing moieties.

In yet another aspect, A can be selected from among H, NH₂, OH, CO₂H, C₁₋₆ alkoxy, and C₁₋₆ alkyls. In some other preferred embodiments, A can be methyl, ethyl, methoxy, ethoxy, H, and OH. A is more preferably methyl or methoxy.

In another preferred embodiment, compounds described herein have the formula:

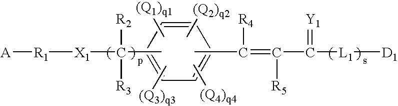

wherein A is a capping group or

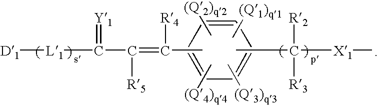

More preferably, compounds described herein can be, for example,

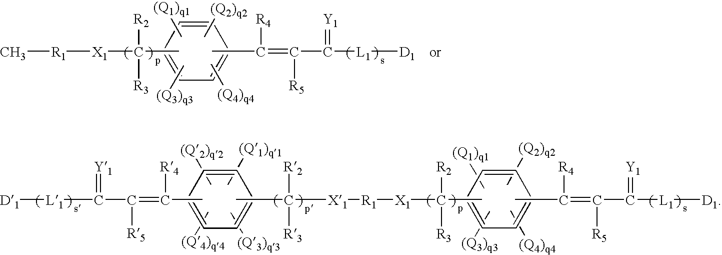

In a further embodiment, the compounds described herein have the formula:

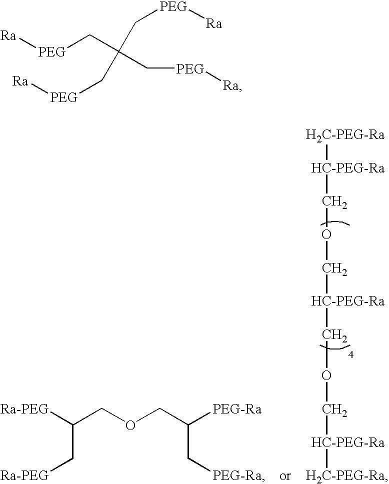

wherein
Ra is

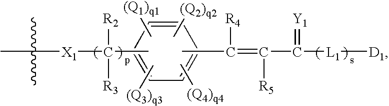

and at least one of D₁ is a targeting group, a diagnostic agent or a biologically active moiety.

Preferably the multi-arm polymer includes at least one targeting group and at least one biologically active moiety. The multi-arm polymeric conjugates containing one or more biologically active moieties are contemplated.

In more preferred embodiments, the sum of (q₁)+(q₂)+(q₃)+(q₄) or the sum of (q′₁)+(q′₂)+(q′₃)+(q′₄) equals to 0 or 1. Compounds of the present invention can be, for example,

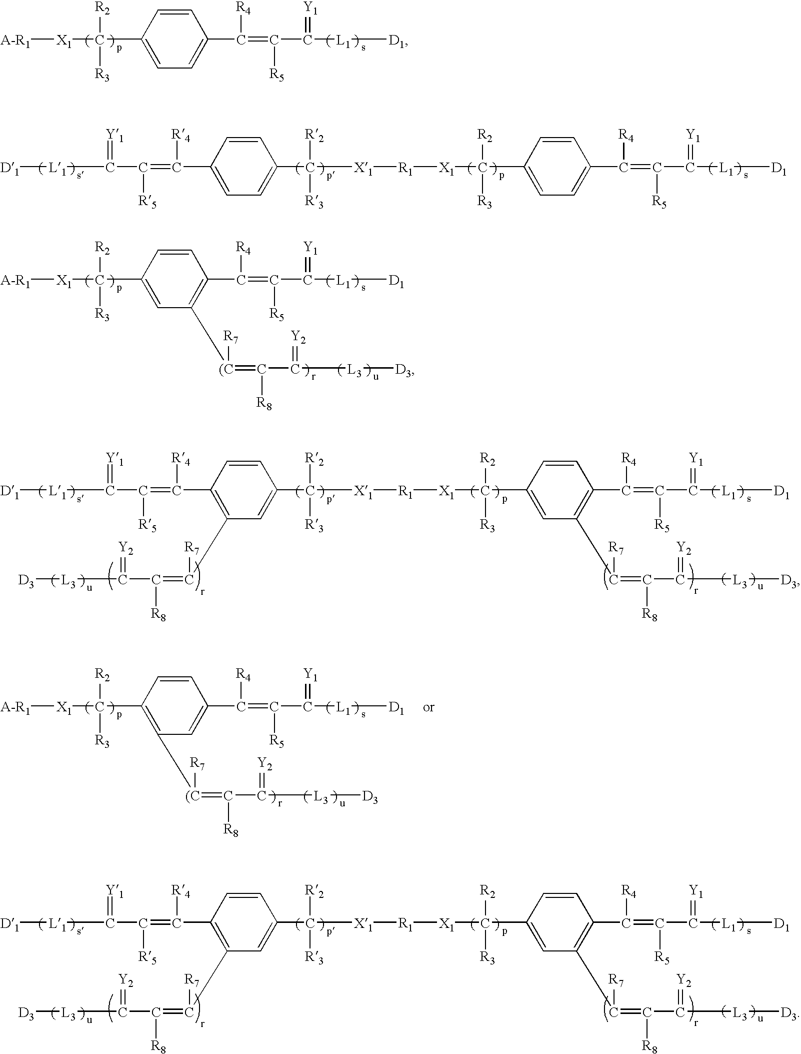

Alternative embodiments can be for example,

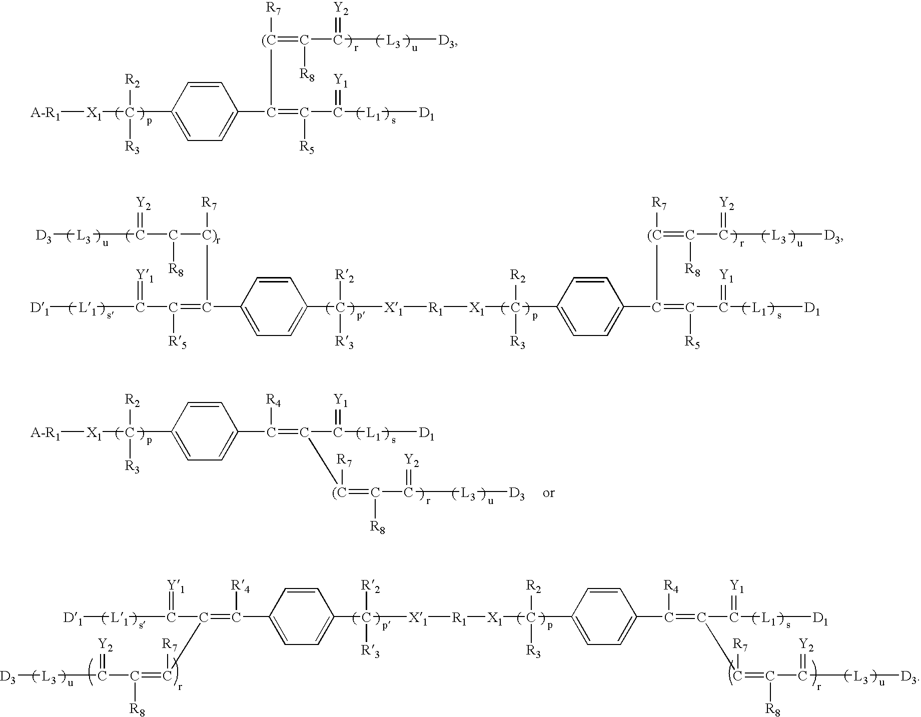

In some preferred embodiments, R₄, R′₄, R₅ and R′₅ are independently hydrogen or CH₃. In some particularly preferred embodiments, R₄, R′₄, R₅ and R′₅ are all hydrogen or CH₃. In other particular embodiments, R₇ and R₈ include hydrogen and CH₃. In yet other particular embodiments, X₁ includes O and NR₆, and Q₁₋₄ include hydrogen, C₁₋₆ alkyls, cycloalkyls, aryls, and aralkyl groups.

In more preferred embodiments, (r) is zero and (u) is 1. Illustrative examples have the formula:

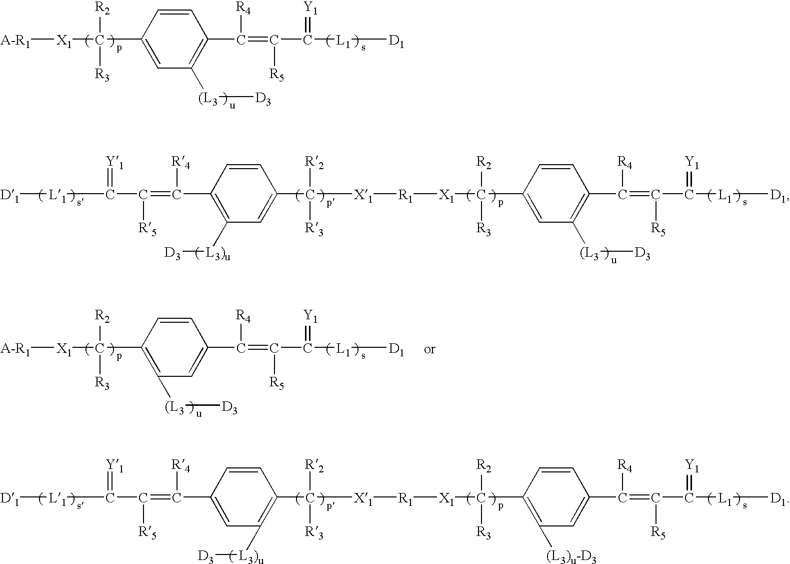

### B. Substantially Non-Antigenic Water-Soluble Polymers

Polymers employed in the compounds described herein are preferably water soluble polymers and substantially non-antigenic such as polyalkylene oxides (PAO's).

In one aspect of the invention, the compounds described herein include a linear, terminally branched or multi-armed polyalkylene oxide. In some preferred embodiments, the polyalkylene oxide includes polyethylene glycol and polypropylene glycol.

The polyalkylene oxide has an average molecular weight from about 2,000 to about 100,000 daltons, preferably from about 5,000 to about 60,000 daltons. The polyalkylene oxide can be more preferably from about 5,000 to about 25,000 or alternatively from about 20,000 to 15 about 45,000 daltons (preferably when small molecular weight compounds having an average molecular weight of less than 1,500 daltons, more preferably 1,200 daltons are conjugated to the polymer). In some particularly preferred embodiments, the compounds described herein include the polyalkylene oxide having an average molecular weight of from about 12,000 to about 20,000 daltons or from about 30,000 to about 45,000 daltons. In one particular embodiment, polymeric portion has a molecular weight of about 12,000 or 40,000 daltons.

The polyalkylene oxide includes polyethylene glycols and polypropylene glycols. More preferably, the polyalkylene oxide includes polyethylene glycol (PEG). PEG is generally represented by the structure:

—O—(CH₂CH₂O)ₙ—

where (n) represents the degree of polymerization for the polymer, and is dependent on the molecular weight of the polymer. Alternatively, the polyethylene glycol (PEG) residue portion of the invention can be selected from among:

—X₁₁—(CH₂CH₂O)ₙ—CH₂CH₂X₁₁—,

—X₁₁—(CH₂CH₂O)ₙ—CH₂C(═Y₁₁)—X₁₁—,

—X₁₁—C(═Y₁₁)—(CH₂)_{n′}—Y₁₂—(CH₂CH₂O)ₙ—CH₂C₂—Y₁₂—(CH₂)_{n′}—C(═Y₁₁)—X₁₁—, and

—X₁₁—(CR₃₁R₃₂)_{n′}—Y₁₂—(CH₂)_{b′}—O—(CH₂CH₂O)ₙ—(CH₂—)_{b′}—Y₁₂—(CR₃ₗR₃₂)_{n′}—X₁₁—,

wherein:

X₁₁ is O, S, SO, SO₂, NR₃₃ or a bond;

Y₁₁ and Y₁₂ are independently O, S, or NR₃₃;

R₃₁₋₃₃ are independently the same moieties which can be used for R₂;

(a′) and (b′) are independently zero or a positive integer, preferably 0-6 and more preferably 1; and

(n) is an integer from about 10 to about 2300.

Branched or U-PEG derivatives are described in U.S. Pat. Nos. 5,643,575, 5,919,455, 6,113,906 and 6,566,506, the disclosure of each of which is incorporated herein by reference. A non-limiting list of such polymers corresponds to polymer systems (i)-(vii) with the following structures:

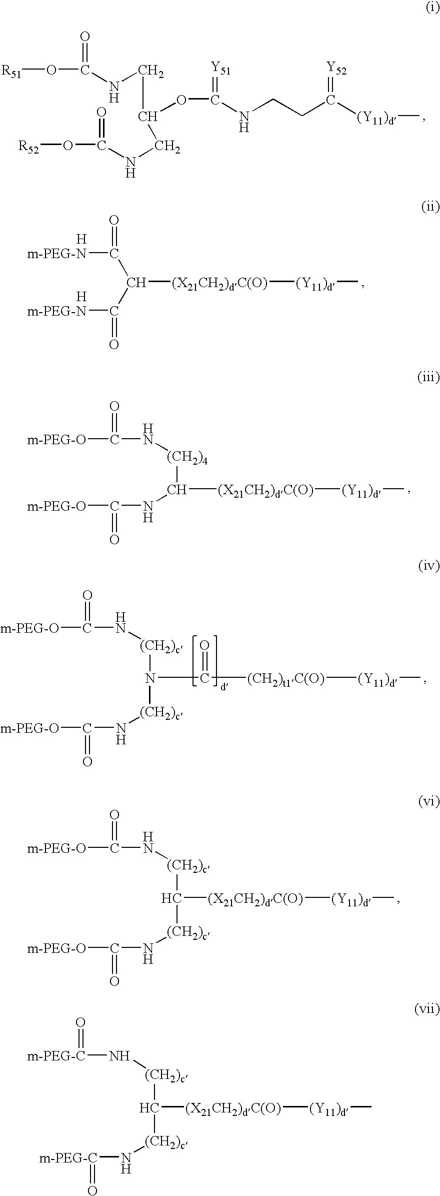

wherein;

R₅₁₋₅₂ are polyalkylene oxide;

Y₁₁ and Y₅₁₋₅₂ are independently O, S or NR₃₃;

X₂₁ is O, NR₆, S, SO or SO₂

(c′) and (t1′) are independently 0 or a positive integer such as 1, 2, 3′ 4 and 5;

(d′) is 0 or 1;

mPEG is methoxy PEG,
wherein PEG is previously defined and a total molecular weight of the polymer portion is from about 2,000 to about 100,000 daltons. R₆ and R₃₃ are previously defined.

In yet another aspect, the polymers include multi-arm PEG-OH or “star-PEG” products such as those described in NOF Corp. Drug Delivery System catalog, Ver. 8, April 2006, the disclosure of which is incorporated herein by reference. See also Shearwater Corporation's 2001 catalog “Polyethylene Glycol and Derivatives for Biomedical Application”, the disclosure of which is incorporated herein by reference. The multi-arm polymer conjugates contain four or more polymer arms and preferably four or eight polymer arms.

For purposes of illustration and not limitation, the multi-arm polyethylene glycol (PEG) residue can be

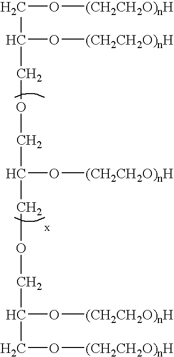

wherein:

(x) is zero and a positive integer, i.e. from about 0 to about 28; and

(n) is the degree of polymerization.

In one particular embodiment of the present invention, the multi-arm PEG has the structure:

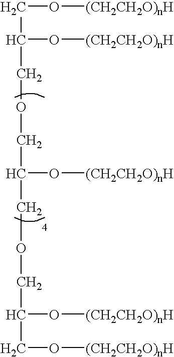

wherein (n) is a positive integer. In one preferred embodiment of the invention, the polymers have a total molecular weight of from about 5,000 Da to about 60,000 Da, and preferably from 20,000 Da to 45,000 Da.

In yet another particular embodiment, the multi-arm PEG has the structure:

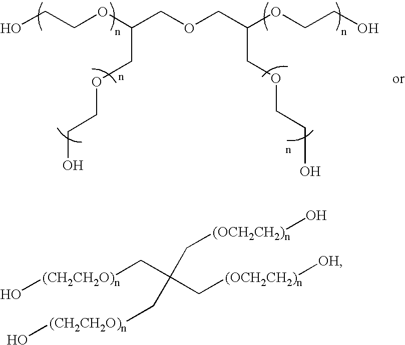

wherein (n) is a positive integer. In one preferred embodiment of the invention, the degree of polymerization for the multi-arm polymer (n) is from about 28 to about 350 to provide polymers having a total molecular weight of from about 5,000 Da to about 60,000 Da, and preferably from 12,000 Da to 45,000 Da. This represents the number of repeating units in the polymer chain and is dependent on the molecular weight of the polymer.

The polymers can be converted into a suitably activated polymer, using the activation techniques described in U.S. Pat. No. 5,122,614 or 5,808,096. Specifically, such PEG can be of the formula:

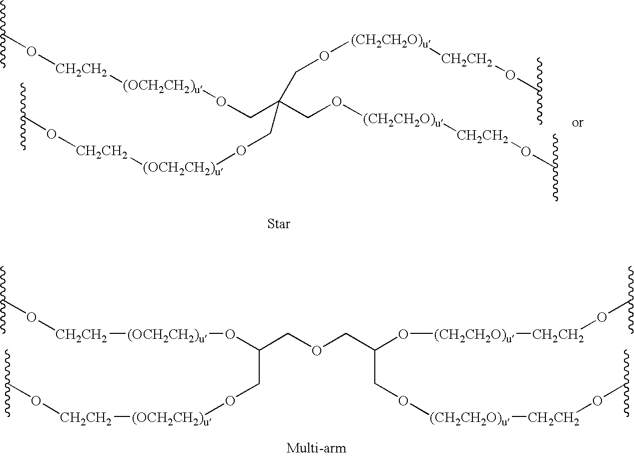

wherein:

(u′) is an integer from about 4 to about 455; and up to 3 terminal portions of the residue is/are capped with a methyl or other lower alkyl.

In some preferred embodiments, all four of the PEG arms can be converted to suitable activating groups for facilitating attachment to aromatic groups. Such compounds prior to conversion include:

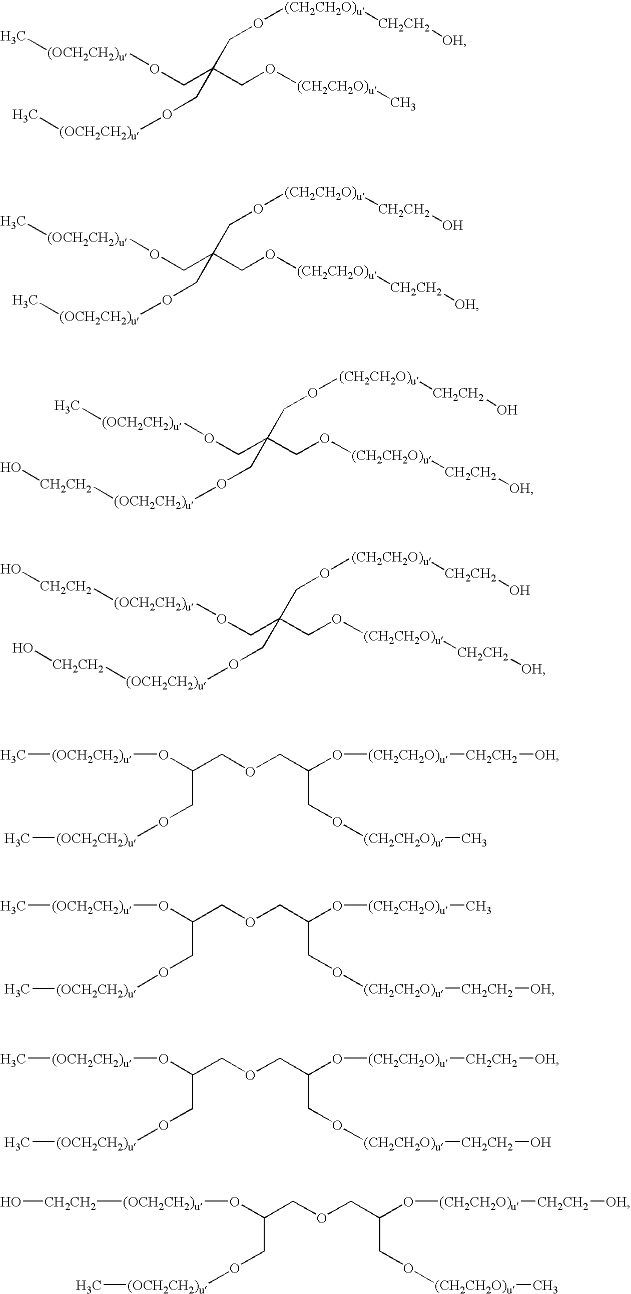

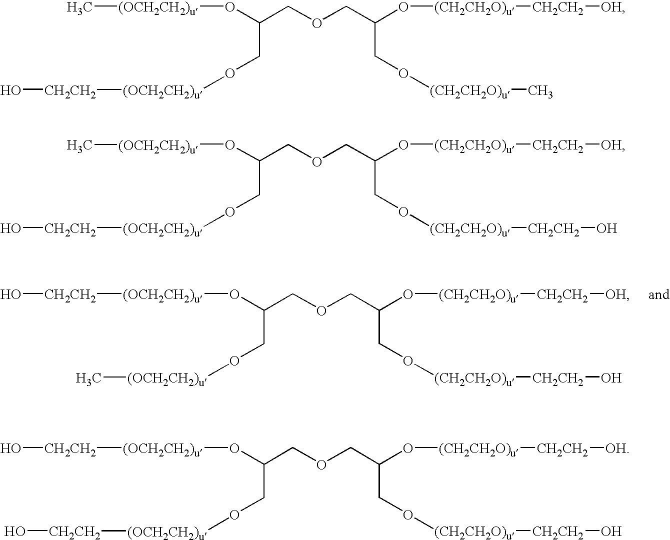

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment, and as an alternative to PAO-based polymers, one or more effectively non-antigenic materials such as dextran, polyvinyl alcohols, carbohydrate-based polymers, hydroxypropylmetharcylamide (HPMA), polyalkylene oxides, and/or copolymers thereof can be used. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

In some aspects, polymers having terminal amine groups can be employed to make the compounds described herein. The methods of preparing polymers containing terminal amines in high purity are described in U.S. patent application Ser. Nos. 11/508,507 and 11/537,172, the contents of each of which are incorporated by reference. For example, polymers having azides react with phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

In alternative aspects, polymers having terminal carboxylic acid groups can be employed in the polymeric delivery systems described herein. Methods of preparing polymers having terminal carboxylic acids in high purity are described in U.S. patent application Ser. No. 11/328,662, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium t-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

### C. Aromatic Moieties

Aromatic moieties (Ar) employed in the compounds described herein include a multi-substituted aromatic or heteroaromatic hydrocarbon. A key feature is that the Ar/Ar′ group is aromatic in nature. Generally, the π electrons must be shared within a “cloud” both above and below the plane of a cyclic molecule. Furthermore, the number of π electrons must satisfy the Hückle rule (4n+2). Those of ordinary skill will realize that a myriad of moieties will satisfy the aromatic requirement of the moiety and thus are suitable for use herein.

In one particular embodiment of the invention, the aromatic moieties include

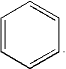

Other suitable aromatic moieties include:

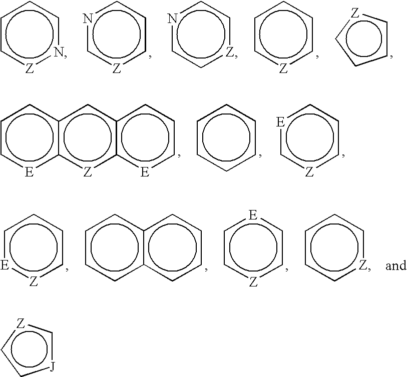

wherein J is O, S, or NR₁₁; E and Z are each independently CR₁₂ or NR₁₃; and R₁₁, R₁₂ and R₁₃ can be selected from among the same moieties which can be used for R₂.

Isomers of the five and six-membered rings are also contemplated as well as benzo- and dibenzo-rings such as anthracine and napthlene and their related congeners are also contemplated within the scope of the invention.

Furthermore, the aromatic or heteroaromatic moieties may optionally be substituted with halogen(s) and/or side chains. All structures suitable for Ar moieties of the present invention are capable of allowing the substituents on the aromatic group to be aligned within the same plane. Ortho, meta and para substituted aromatic rings can be used.

### D. Bifunctional Linkers

Bifunctional linkers include amino acids, amino acid derivatives and peptides. The amino acids can be among naturally occurring and non-naturally occurring amino acids. Derivatives and analogs of the naturally occurring amino acids, as well as various art-known non-naturally occurring amino acids (D or L), hydrophobic or non-hydrophobic, are also contemplated to be within the scope of the invention. A suitable non-limiting list of the non-naturally occurring amino acids includes 2-aminoadipic acid, 3-aminoadipic acid, beta-alanine, beta-aminopropionic acid, 2-aminobutyric acid, 4-aminobutyric acid, piperidinic acid, 6 aminocaproic acid, 2-aminoheptanoic acid, 2-aminoisobutyric acid, 3-aminoisobutyric acid, 2 aminopimelic acid, 2,4-aminobutyric acid, desmosine, 2,2-diaminopimelic acid, 2,3 diaminopropionic acid, N-ethylglycine, N-ethylasparagine, 3-hydroxyproline, 4-hydroxyproline, isodesmosine, allo-isoleucine, N-methylglycine, sarcosine, N-methyl-isoleucine, 6-N-methyl-lysine, N-methylvaline, norvaline, norleucine, and ornithine. Some preferred amino acid residues are selected from glycine, alanine, methionine and sarcosine, and more preferably, glycine.

Alternatively, L₁, L₁′ and L₃ can be selected from among

—[C(═O)]ᵥ(CR₂₂R₂₃)ₜ[C(═O)]_{v′}—,

—[C(═O)]ᵥ(CR₂₂R₂₃)ₜ—O[C(═O)]_{v′}—,

—[C(═O)]ᵥ(CR₂₂R₂₃)ₜ—NR₂₆[C(═O)]_{v′}—,

—[C(═O)]ᵥO(CR₂₂R₂₃)ₜ[C(═O)]_{v′}—,

—[C(═O)]ᵥO(CR₂₂R₂₃)ₜO[C(═O)]_{v′}—,

—[C═O)]ᵥO(CR₂₂R₂₃)ₜNR₂₆[C(═O)]_{v′}—,

—[C(—O)]ᵥNR₂₁(CR₂₂R₂₃)ₜ[C(O)]_{v′}—,

—[C(═O)]ᵥNR₂₁(CR₂₂R₂₃)ₜO[C(═O)]_{v′}—,

—[C(═O)]ᵥNR₂₁(CR₂₂R₂₃)ₜNR₂₆[C(═O)]_{v′}—,

—[C(═O)]_{b}(CR₂₂R₂₃)ₜO—(CR₂₈R₂₉)ₜ′[C(═O)]_{v′}—,

—[C(═O)]ᵥ(CR₂₂R₂₃)ₜNR₂₆—(CR₂₈R₂₉)_{t′}[C(═O)]_{v′}—,

—[C(═O)]ᵥ(CR₂₂R₂₃)ₜS—(CR₂₈R₂₉)_{t′}[C(—O)]_{v′}—,

—[C(═O)]ᵥO(CR₂₂R₂₃)ₜO—(CR₂₈R₂₉)_{t′}[C(═O)]_{v′}—,

—[C(═O)]ᵥO(CR₂₂R₂₃)ₜNR₂₆—(CR₂₈R₂₉)_{t′}[C(═O)]_{v′}—,

—[C(═O)]ᵥO(CR₂₂R₂₃)ₜS—(CR₂₈R₂₉)_{t′}[C(═O)]_{v′}—,

—[C(═O)]ᵥNR₂₁(CR₂₂R₂₃)ₜO—(CR₂₈R₂₉)_{t′}[C(═O)]_{v′}—,

—[C(O)]ᵥNR₂ₗ(CR₂₂R₂₃)ₜNR₂₆—(CR₂₈R₂₉)_{t′}[C(═O)]_{v′}—,

—[C(═O)]ᵥNR₂₁(CR₂₂R₂₃)ₜS—(CR₂₈R₂₉)ₜ[C(═O)]_{v′}—,

—[C(═O)]ᵥ(CR₂₂R₂₃CR₂₈R₂₉O)ₜNR₂₆[C(═O)]_{v′}—,

—[C(—O)]ᵥ(CR₂₂R₂₃CR₂₈R₂₉O)ₜ[C(═O)]_{v′}—,

—[C(═O)]ᵥO(CR₂₂R₂₃CR₂₈R₂₉O)ₜNR₂₆[C(—O)]_{v′}—,

—[C(═O)]ᵥO(CR₂₂R₂₃CR₂₈R₂₉O)ₜ[C(═O)]_{v′}—,

—[C(═O)]ᵥNR₂₁(CR₂₂R₂₃CR₂₈R₂₉O)ₜNR₂₆[C(—O)]_{v′}—,

—[C(═O)]ᵥNR₂₁(CR₂₂R₂₃CR₂₈R₂₉O)ₜ[C(═O)]_{v′}—,

—[C(—O)]ᵥ(CR₂₂R₂₃CR₂₈R₂₉O)ₜ(CR₂₄R₂₅)_{t′}[C(═O)]_{v′}—,

—[C(═O)]ᵥO(CR₂₂R₂₃CR₂₈R₂₉O)ₜ(CR₂₄R₂₅)_{t′}[C(═O)]_{v′}—,

—[C(═O)]ᵥNR₂₁(CR₂₂R₂₃CR₂₈R₂₉O)(CR₂₄R₂₅)_{t′}[C(═O)]_{v′}—,

—[C(═O)]ᵥ(CR₂₂R₂₃CR₂₈R₂₉O)ₜ(CR₂₄R₂₅)_{t′}O[C(—O)]_{v′}—,

—[C(═O)]ᵥ(CR₂₂R₂₃)ₜ(CR₂₄R₂₅CR₂₈R₂₉O)_{t′}[C(═O)]_{v′}—,

—[C(═O)]ᵥ(CR₂₂R₂₃)ₜ(CR₂₄R₂₅(CR₂₈R₂₉O)ₜ—NR₂₆[C(═O)]_{v′}—,

—[C(═O)]ᵥO(CR₂₂R₂₃CR₂₈R₂₉O)ₜ(CR₂₄R₂₅)_{t′}O[C(═O)]_{v′}—,

—[C(═O)]ᵥO(CR₂₂R₂₃)ₜ(CR₂₄R₂₅CR₂₈R₂₉O)_{t′}[C(═O))]_{v′}—,

—[C(═O)]ᵥO(CR₂₂R₂₃)ₜ(CR₂₄CR₂₅CR₂₈R₂₉O)ₜ—NR₂₆[C(—O)]_{v′}—,

—[C(═O)]ᵥNR₂₁(CR₂₂R₂₃CR₂₈R₂₉O)ₜ(CR₂₄R₂₅)_{t′}O[C(═O)]_{v′}—,

—[C(═O)]ᵥNR₂₁(CR₂₂R₂₃)ₜ(CR₂₄R₂₅CR₂₈R₂₉O)_{t′}[C(═O)]_{v′}—,

—[C(═O)]ᵥNR₂₁(CR₂₂R₂₃)ₜ(CR₂₄R₂₅CR₂₈R₂₉O)_{t′}NR₂₆[C(═O)]_{v′}—,

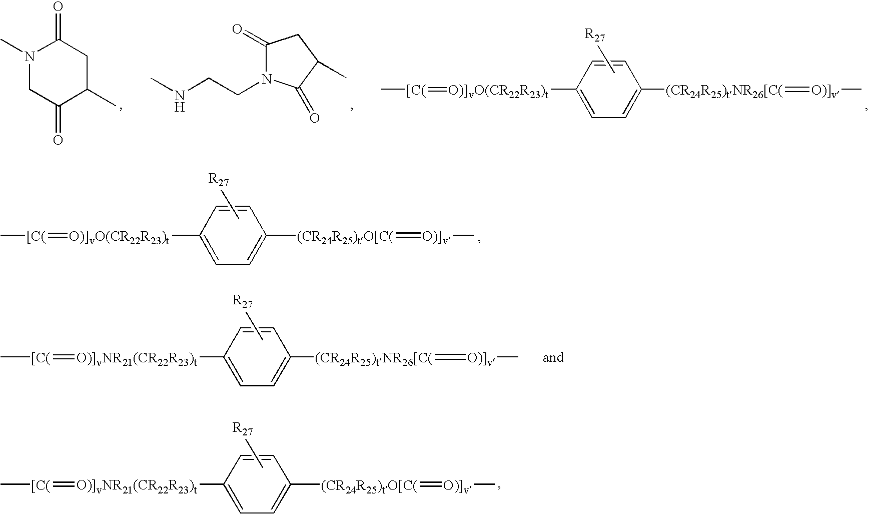

wherein:

R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆(alkoxy, phenoxy and C₁₋₆ heteroalkoxy;

(t) and (t′) are independently zero or a positive integer, preferably from about 0 to about 10 (e.g., 0, 1, 2, 3, 4, 5, 6, 7, 8, 9 or 10), more preferably from about 0 to about 6 (e.g., 0, 1, 2, 3, 4, 5 or 6), and yet more preferably 0, 1 or 2; and

(v) and (v′) are independently zero or 1.

For purposes of the present invention, C(R₂₄)(R₂₅) is the same or different when (t) or (t′) is equal to or greater than 2.

Preferably, L₁, L₁′ and L₃ can be selected from among:

—[C(═O)]ᵣ₁NH(CH₂)₂CH═N—NHC(═O)—(CH₂)₂—,

—[C(═O)]ᵣ₁NH(CH₂)₂(CH₂CH₂O)₂(CH₂)₂NH[C(═O)]_{r1′}—,

—[C(═O)]ᵣ₁NH(CH₂CH₂)(CH₂CH₂O)₂NH[C(═O)]_{r1′}—,
—[C(═O)]ᵣ₁NH(CH₂CH₂)ₛ₁NH(CH₂CH₂)_{s1′}[C(═O)]_{r1′}—,

—[C(═O)]ᵣ₁NH(CH₂CH₂)ₛ₁S(CH₂CH₂)_{s1′}[C(═O)]_{r1′}—,

—[C(O)]ᵣ₁NH(CH₂CH₂)(CH₂CH₂O)[C(═O)]_{r1′}—,

—[C(O)]ᵣ₁NH(CH₂CH₂)ₛ₁O(CH₂CH₂)_{s1′}[C(═O)]_{r1′}—,

—[C(═O)]ᵣ₁NH(CH₂CH₂O)(CH₂)NH[C(═O)]_{r1′}—,

—[C(—O)]ᵣ₁NH(CH₂CH₂O)₂(CH₂)[C(═O)]_{r1′}—,

—[C(═O)]ᵣ₁NH(CH₂CH₂O)ₛ₁(CH₂)_{s1′}[C(═O)]_{r1′}—,

—[C(═O)]ᵣ₁NHCH₂CH₂NH[C(═O)]_{r1′}—,

—[C(═O)]ᵣ₁NH(CH₂CH₂)₂O[C(═O)]_{r1′}—,

—[C(═O)]ᵣ₁NH(CH₂CH₂O)[C(═O)]_{r1′}—,

—[C(═O)]ᵣ₁NH(CH₂CH₂O)₂[C(═O)]_{r1′}—,

—[C(═O)]ᵣ₁NH(CH₂)₃[C(═O)]_{r1′}—,

—[C(O)]ᵣ₁O(CH₂CH₂O)₂(CH₂)[C(═O)]_{r1′}—,

—[C(═O)]ᵣ₁O(CH₂)₂NH(CH₂)₂[C(═O)]_{r1′}—,

—[C(═O)]ᵣ₁O(CH₂CH₂O)₂NH[C(═O)]_{r1′}—,

—[C(═O)]ᵣ₁O(CH₂)₂—O—(CH₂)₂[C(═O)]_{r1′}—,

—[C(═O)]ᵣ₁O(CH₂)₂S(CH₂)₂[C(═O)]_{r1′}—,

—[C(═O)]ᵣ₁O(CH₂CH₂)NH[C(═O)]_{r1′}—,

—[C(═O)]ᵣ₁O(CH₂CH₂)O[C(═O)]_{r1′}—,

—[C(═O)]ᵣ₁O(CH₂)₃NH[C(═O)]_{r1′}—,

—[C(═O)]ᵣ₁O(CH₂)₃O[C(═O)]_{r1′}—,

—[C(═O)]ᵣ₁O(CH₂)₃[C(═O)]_{r1′}—,

—[C(═O)]ᵣ₁CH₂NHCH₂[C(═O)]_{r1′}—,

—[C(═O)]ᵣ₁CH₂OCH₂[C(═O)]_{r1′}—,

—[C(═O)]ᵣ₁CH₂SCH₂[C(═O)]_{r1′}—,

—[C(═O)]ᵣ₁S(CH₂)₃[C(═O)]_{r1′}—,

—[C(═O)]ᵣ₁(CH₂)₃[C(═O)]_{r1′}—,

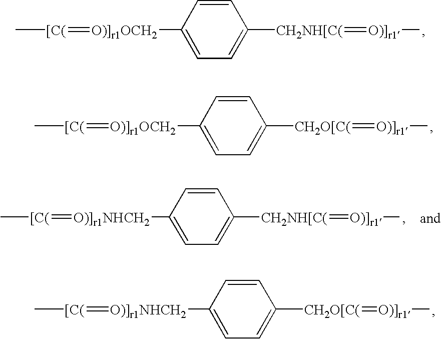

wherein

(r1) and (r1′) are independently zero or 1; and

(s1) and (s1′) are independently zero or a positive integer, preferably from about 0 to about 4 (e.g., 0, 1, 2, 3, or 4), more preferably 0, 1 or 2,

provided that both (r1) and (r1′) are not zero simultaneously.

In yet further alternative aspects of the invention, L₁. L′₁ and L₃ include:

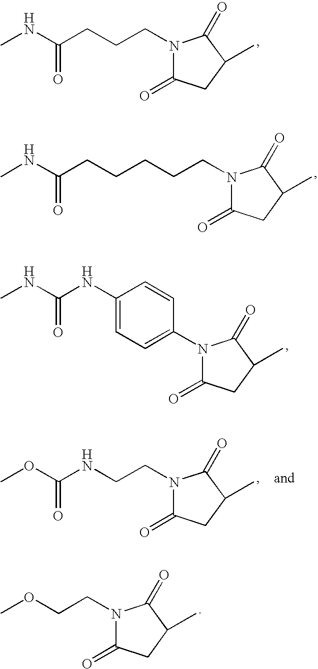

In a further embodiment and as an alternative, L₁, L′₁ and L₃ include structures corresponding to those shown above but having vinyl, residues of sulfone, amino, carboxy, mercapto, hydrazide, carbazate and the like instead of maleimidyl. These bifunctional groups allow a second agent to be directly conjugated and therefore eliminate the need of attaching a functional group for conjugating to a second agent.

### E. D₁, D′₁ and D₃ Groups

### 1. Leaving Groups and Functional Groups

In some aspects, suitable leaving groups include, without limitations halogen (Br, Cl), activated carbonate, carbonyl imidazole, cyclic imide thione, isocyanate, N-hydroxysuccinimidyl, para-nitrophenoxy, N-hydroxphtalmide, N-hydroxybenzotriazolyl, imidazole, tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, pentafluorophenoxy, 1,3,5-trichlorophenoxy, and 1,3,5 trifuorophenoxy or other suitable leaving groups as will be apparent to those of ordinary skill.

For purposes of the present invention, leaving groups are to be understood as those groups which are capable of reacting with a nucleophile found on the desired target, i.e. a biologically active moiety, a diagnostic agent, a targeting moiety, a bifunctional spacer, intermediate, etc. The targets thus contain a group for displacement, such as OH, NH₂ or SH groups found on proteins, peptides, enzymes, naturally or chemically synthesized therapeutic molecules such as doxorubicin, and spacers such as mono-protected diamines.

In some preferred embodiments, functional groups to link the polymeric transport systems to biologically active moieties include maleimidyl, vinyl, residues of sulfone, amino, carboxy, mercapto, hydrazide, carbazate and the like which can be further conjugated to a biologically active group.

In particularly preferred embodiments of the invention, D₁, D′₁ and D₃ can be selected from among OH, methoxy, tert-butoxy, N-hydroxysuccinimidyl and maleimidyl.

### 2. Biologically Active Moieties

A wide variety of biologically active moieties can be attached to the activated polymers described herein. The biologically active moieties include pharmaceutically active compounds, enzymes, proteins, oligonucleotides, antibodies, monoclonal antibodies, single chain antibodies and peptides. In addition, the activated polymer of the invention can further contain a biologically active moiety as D₁, D′₁ and D₃ which includes amine-, hydroxyl-, or thiol-containing compounds. A non-limiting list of such suitable compounds includes organic compounds, enzymes, proteins, polypeptides, antibodies, monoclonal antibodies, single chain antibodies or oligonucleotides, etc.

For purposes of the present invention, it shall be understood to mean that the pharmaceutically active compounds include small molecular weight molecules. Typically, the pharmaceutically active compounds have a molecular weight of less than about 1,500 daltons and optionally derivatized with SH containing moiety to provide reactive site for conjugation with polymer.

In some aspects of the invention, biologically active moieties include amine-, hydroxyl-, or thiol-containing compounds. A non-limiting list of such suitable compounds includes organic compounds, enzymes, proteins, polypeptides, antibodies, monoclonal antibodies, single chain antibodies or oligonucleotides, etc. Organic compounds include, without limitation, moieties such as camptothecin and analogs (e.g., SN38 and irinotecan), and related topoisomerase I inhibitors, taxanes and paclitaxel derivatives, nucleosides including AZT, anthracycline compounds including daunorubicin, doxorubicin; p-aminoaniline mustard, melphalan, Ara-C (cytosine arabinoside) and related anti-metabolite compounds, e.g., gemcitabine, etc. Alternatively, biologically active moieties can include cardiovascular agents, anti-neoplastic, anti-infective, anti-fungal such as nystatin and amphotericin B, anti-anxiety agents, gastrointestinal agents, central nervous system-activating agents, analgesic, fertility agents, contraceptive agents, anti-inflammatory agents, steroidal agents, anti-urecemic agents, vasodilating agents, and vasoconstricting agents, etc. It is to be understood that other biologically active materials not specifically mentioned but having suitable amine-, hydroxyl or thiol-containing, groups are also intended and are within the scope of the present invention.

In another aspect of the invention, the biologically active compounds are suitable for medicinal or diagnostic use in the treatment of animals, e.g., mammals, including humans, for conditions for which such treatment is desired.

The only limitations on the types of the biologically active moieties suitable for inclusion herein is that there is available at least one amine-, hydroxyl-, or thiol-containing position which can react and link with a carrier portion and that there is not substantial loss of bioactivity in the form of conjugated to the polymeric delivery systems described herein. Alternatively, parent compounds suitable for incorporation into the polymeric transport conjugate compounds of the invention, may be active after hydrolytic release from the linked compound, or not active after hydrolytic release but which will become active after undergoing a further chemical process/reaction. For example, an anticancer drug that is delivered to the bloodstream by the polymeric transport system, may remain inactive until entering a cancer or tumor cell, whereupon it is activated by the cancer or tumor cell chemistry, e.g., by an enzymatic reaction unique to that cell.

A further aspect of the invention provides the conjugate compounds optionally prepared with a diagnostic tag linked to the polymeric delivery system described herein, wherein the tag is selected for diagnostic or imaging purposes. Thus, a suitable tag is prepared by linking any suitable moiety, e.g., an amino acid residue, to any art-standard emitting isotope, radio-opaque label, magnetic resonance label, or other non-radioactive isotopic labels suitable for magnetic resonance imaging, fluorescence-type labels, labels exhibiting visible colors and/or capable of fluorescing under ultraviolet, infrared or electrochemical stimulation, to allow for imaging tumor tissue during surgical procedures, and so forth. Optionally, the diagnostic tag is incorporated into and/or linked to a conjugated therapeutic moiety, allowing for monitoring of the distribution of a therapeutic biologically active material within an animal or human patient.

In yet a further aspect of the invention, the inventive tagged conjugates are readily prepared, by art-known methods, with any suitable label, including, e.g., radioisotope labels. Simply by way of example, these include ¹³¹Iodine, ¹²⁵Iodine, ^{99m}Technetium and/or ¹¹¹Indium to produce radioimmunoscintigraphic agents for selective uptake into tumor cells, in vivo. For instance, there are a number of art-known methods of linking peptide to Tc-99m, including, simply by way of example, those shown by U.S. Pat. Nos. 5,328,679; 5,888,474; 5,997,844; and 5,997,845, incorporated by reference herein.

### 3. Targeting Groups

In some aspects, the compounds described herein include targeting groups. The targeting groups include receptor ligands, an antibodies or antibody fragments, single chain antibodies, targeting peptides such as cell adhesion peptides and cell penetrating peptides (CPPs), targeting carbohydrate molecules or lectins. Targeting groups enhance binding or uptake of the compounds described herein a target tissue and cell population. For example, a non-limiting list of targeting groups includes vascular endothelial cell growth factor, FGF2, somatostatin and somatostatin analogs, transferrin melanotropin, ApoE and ApoE peptides, von Willebrand's Factor and von Willebrand's Factor peptides; adenoviral fiber protein and adenoviral fiber protein peptides; PD1 and PD1 peptides, EGF and EGF peptides, RGD peptides, folate, etc.

Other suitable targeting groups include selectin, TAT, penetratin, and Arg₉.

In a further aspect of the invention, the targeting groups can be optionally labeled with biotin, fluorescent compounds, radio-labeled compounds by art-known methods.

### F. Synthesis of the Polymeric Delivery Systems

Generally, the methods of preparing the compounds described herein include reacting a polymer with an aromatic acid ester to form a polymer aromatic acid. In one aspect of the invention, methods of preparing compounds described herein include

reacting a compound of Formula (II):

A₁-R₁—X₁-M₁ (II)

with a compound of Formula (III):

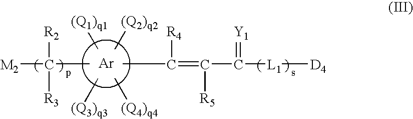

under conditions sufficient to form a compound of Formula (IV):

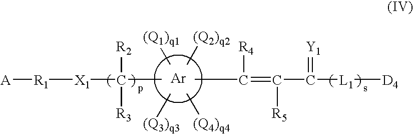

wherein:

A₁ is a capping group or M₁-X′₁-;

A is a capping group or

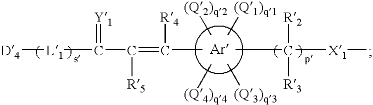

R₁ is a substantially non-antigenic water-soluble polymer;

M₁ is a leaving group;

M₂ is —OH, —SH or —NHR₄₁;

D₄ and D′₄ are independently selected from the group consisting of hydrogen, OH, OR₄₂, functional groups and leaving groups, targeting groups, diagnostic agents and biologically active moieties;

Ar and Ar′ are independently an aryl or heteroaryl moiety;

X₁ and X′₁, are independently O, S, SO, SO₂, NR₆ or a bond;

Y₁ and Y′₁ are independently O, S, or NR₆;

L₁ and L′₁ are independently selected bifunctional linkers;

R₂, R′₂, R₃, R′₃, R₆ and R₄₁ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₈ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy;

R₄₂ is C₁₋₆ alkyl;

(p) and (p′) are independently 0 or a positive integer, preferably from about 0 to about 10 and more preferably from about 0 to about 4, and most preferably 0 or 1;

(q₁), (q′₁), (q₂), (q′₂), (q₃), (q′₃), (q₄), (q′₄), (s) and (s′) are independently zero or one:

R₄, R′₄, R₅, R′₅, Q₁₋₄ and Q′₁₋₄ are independently selected from the same moieties which can be used for R₂ or each can be

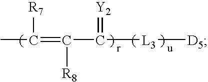

wherein,
R₇ and R₈ are independently selected from the same moieties which can be used
Y₂ is O, S, NR₆;
L₃ is a bifunctional linker;
(r) and (u) are zero or one; and
(u) is zero or a positive integer; and
D₅ is selected from the group consisting of hydrogen, OH, OR₄₂, functional groups and leaving groups, targeting groups, diagnostic agents and biologically active moieties; and
   provided that (r) is not zero when (u) is zero.

In one preferred aspect, X₁ and X′₁ are independently O, S, SO or SO₂ when (p) is zero.

In alternative aspects, the compounds described herein are selected such that the sum of (q₁)+(q₂)+(q₃)+(q₄) is not zero (e.g., preferably 1), and at least one (e.g., one, two or three) of R₄, R′₄, R₅, R′₅, Q₁₋₄ and Q′₁₋₄ is

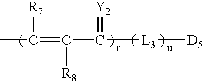

wherein D₅ is selected from the group consisting of leaving groups, functional groups, targeting groups, diagnostic agents and biologically active moieties. With this aspect, the leaving group is preferably selected from among N-hydroxysuccinimidyl, para-nitrophenoxy, ortho-nitrophenoxy and C₁-C₆ alkyloxy, and the functional group is preferably selected from among maleimidyl, vinyl, and residues of sulfone.

In certain aspects of the invention, when (p) is zero, there are preferably a sufficient number of atoms, e.g., more than five or six atoms, present between X₁/X′₁ and C(═Y₁)/C(═Y′₁), so that a releasable cyclic moiety is not formed. For example, when (p) is zero, the polymeric portion attached to the phenyl ring would not be in an ortho position in relation to D₄, D′₄ or D₅.

The leaving group M₁ includes halogen (Br, Cl), activated carbonate, carbonyl imidazole, cyclic imide thione, isocyanate, N-hydroxysuccinimidyl, para-nitrophenoxy, N-hydroxyphtalimide, N-hydroxybenzotriazolyl, imidazole, tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, imidazole, pentafluorophenoxy, 1,3,5-trichlorophenoxy, and 1,3,5-trifluorophenoxy or other suitable leaving groups that is apparent to those of ordinary skill in the art.

The resulting compounds of Formula (IV) can be deprotected to form a polymer-aromatic acid. The polymer-aromatic acids are further activated with an amine or a hydroxyl containing compound. Alternatively/additionally, a bifunctional group can be attached to the aromatic moiety to provide a functional group. The functional groups can be further conjugated to a biologically active moiety, or a targeting moiety.

Alternatively, it is also contemplated that methods can include reacting a polymer with an aromatic moiety containing a leaving group to form a polymer-aromatic acid.

Attachment of the bifunctional group to the polymer portion is preferably carried out in the presence of a coupling agent. A non-limiting list of suitable coupling agents include 1,3-diisopropylcarbodiimide (DIPC), any suitable dialkyl carbodiimides, 2-halo-1-alkyl-pyridinium halides, (Mukaiyama reagents), 1-(3-dimethylaminopropyl)-3-ethyl carbodiimide (EDC) propane phosphonic acid cyclic anhydride (PPACA), and phenyl dichlorophosphates, etc. which are available, for example from commercial sources such as Sigma-Aldrich Co., or synthesized using known techniques.

Preferably, the reactions are carried out in an invert solvent such as methylene chloride, chloroform, DMF or mixtures thereof. The reactions can be preferably conducted in the presence of a base, such as dimethylaminopyride (DMAP), diisopropylethlylamine, pyridine, triethylamine, etc. to neutralize any acids generated. The reactions can be carried out at a temperature from about 0° C. up to about 22° C. (room temperature).

Some particular embodiments prepared by the methods described herein include,

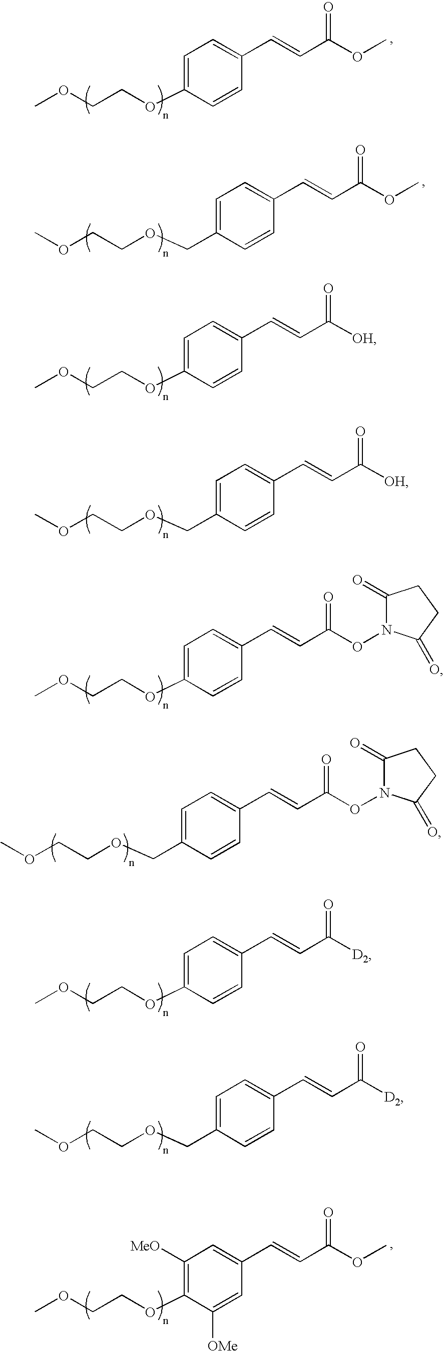

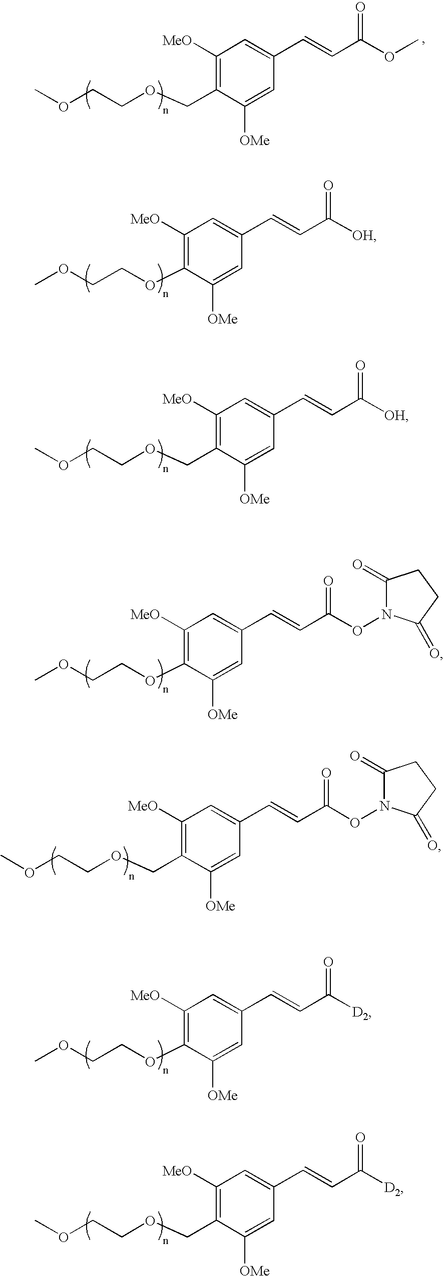

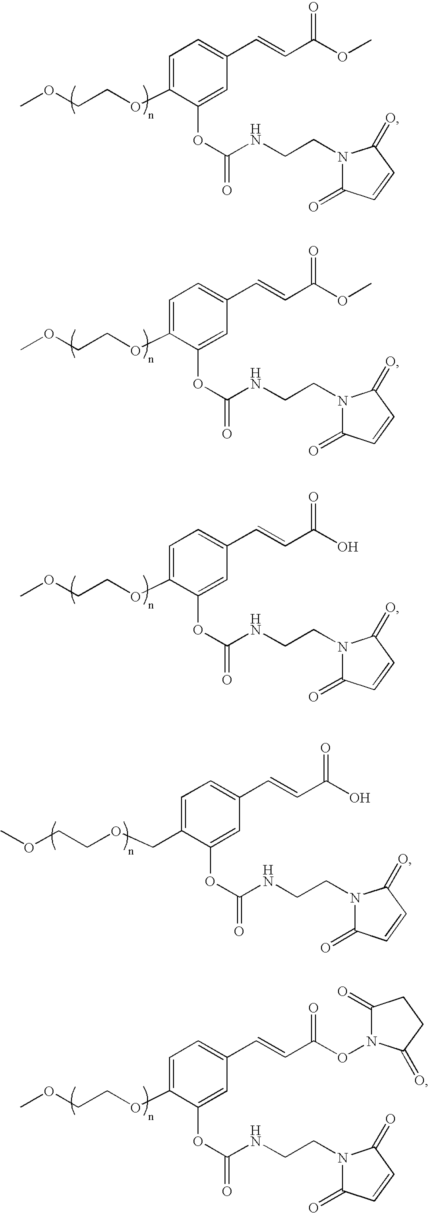

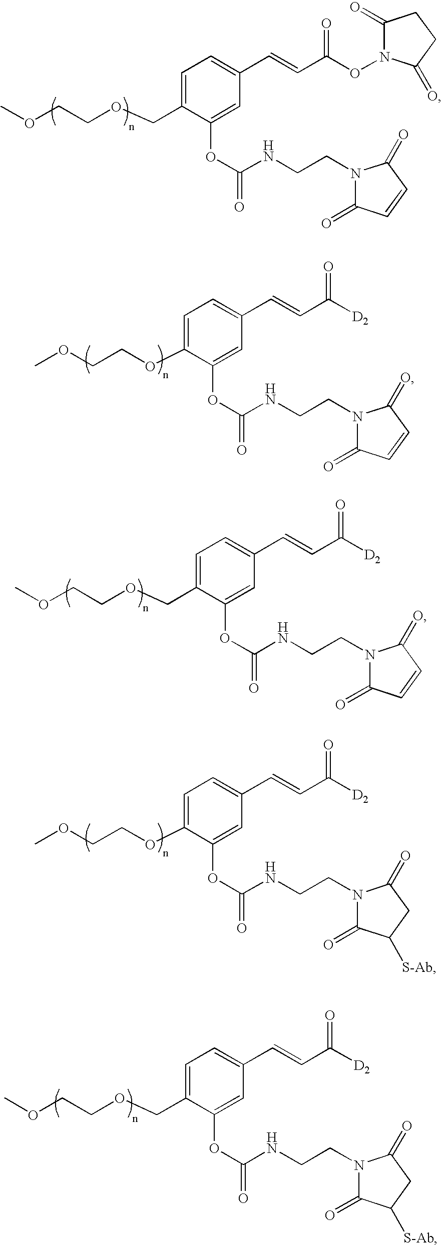

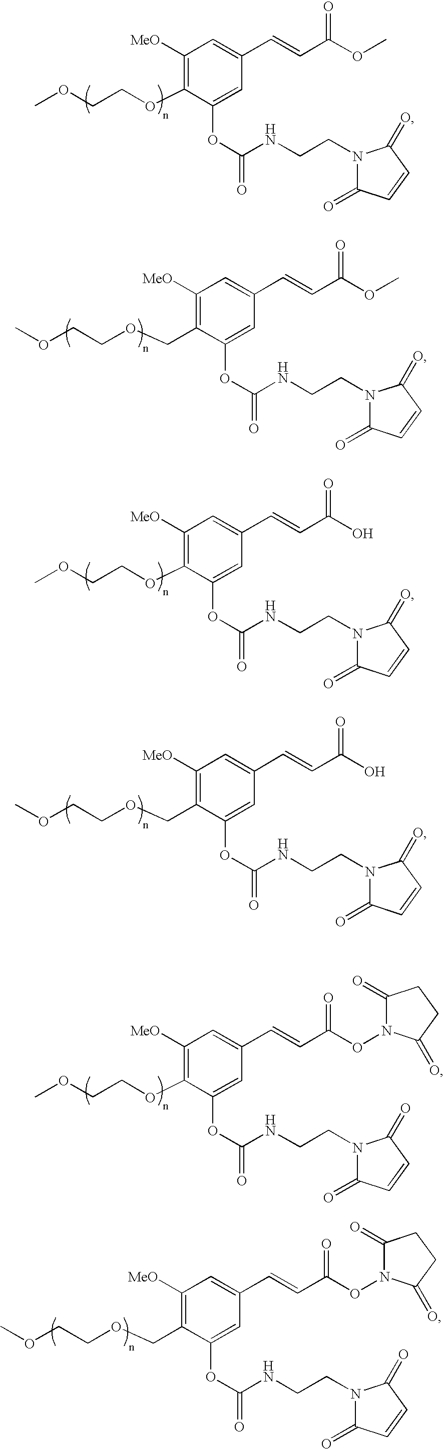

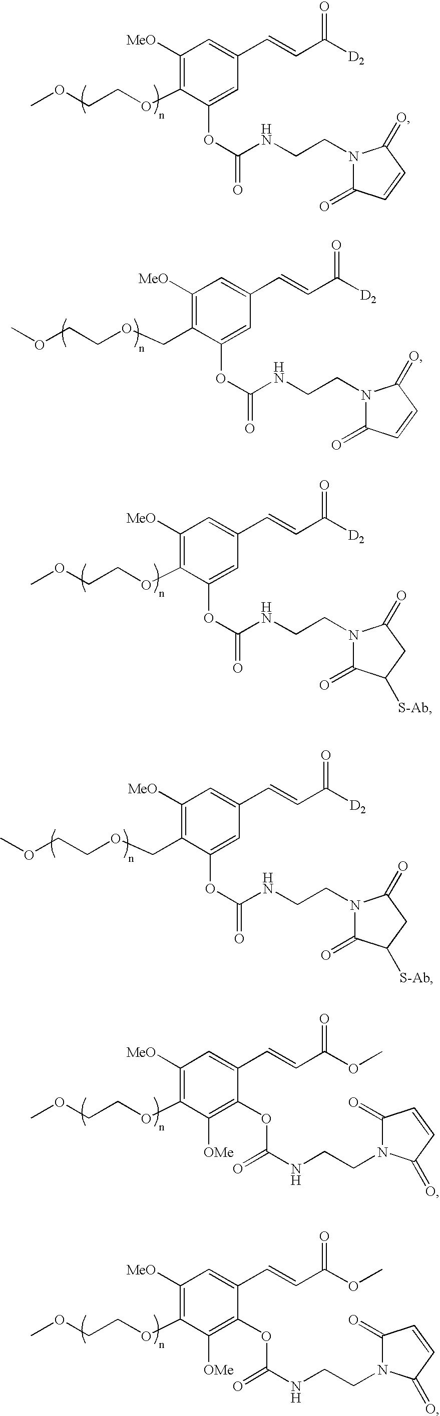

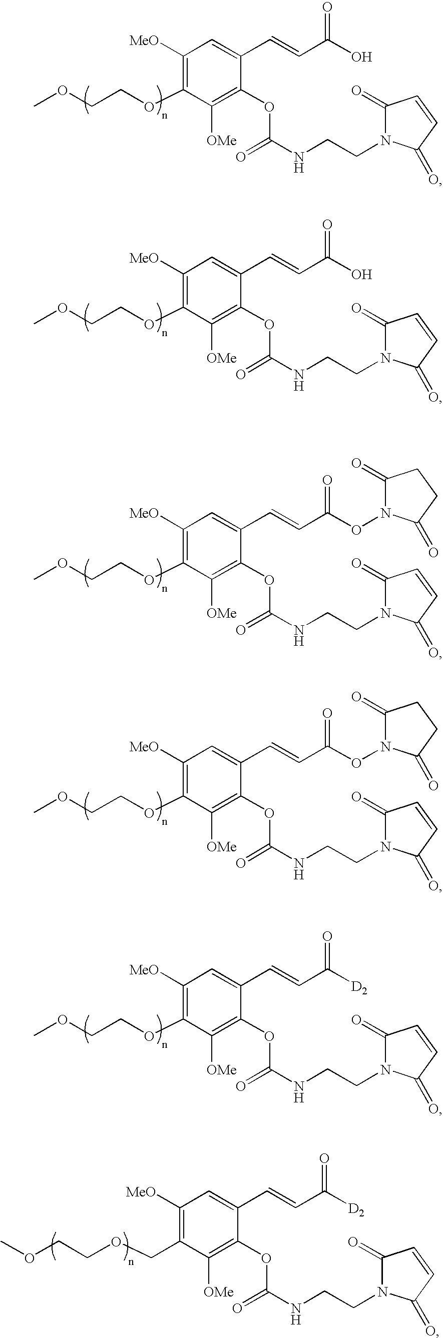

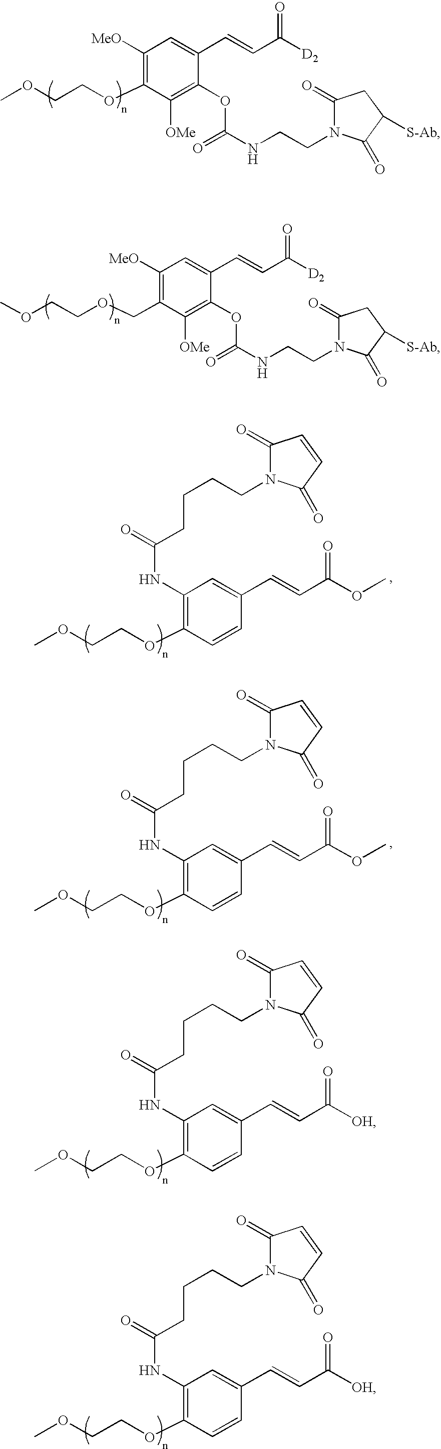

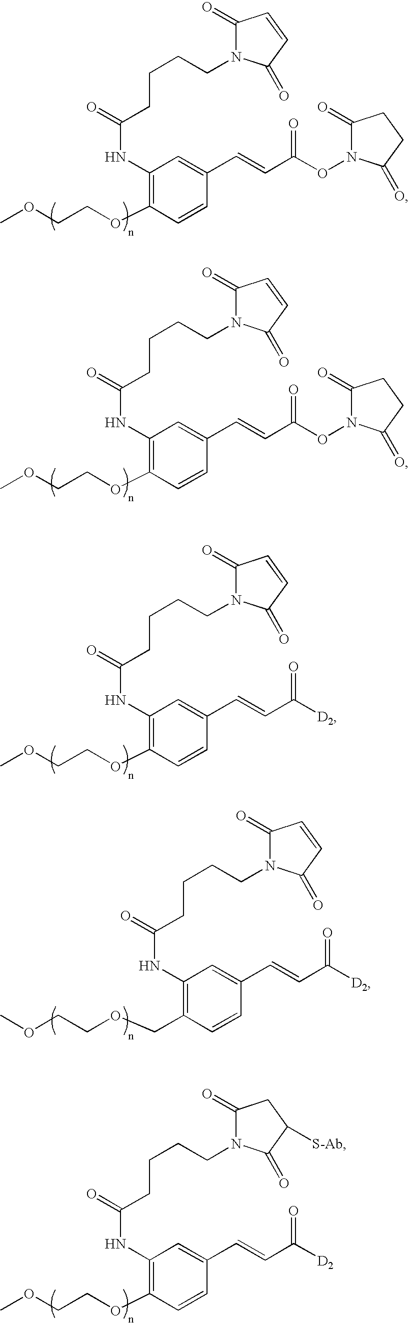

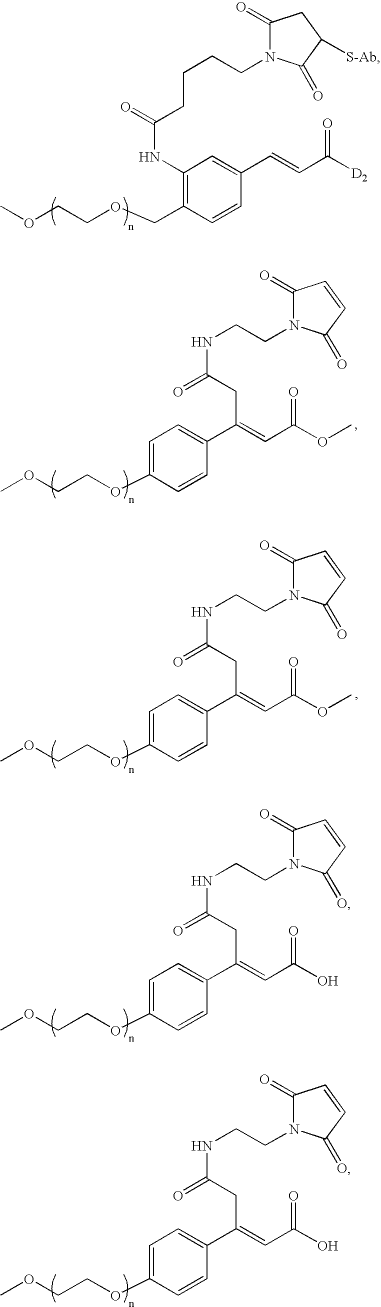

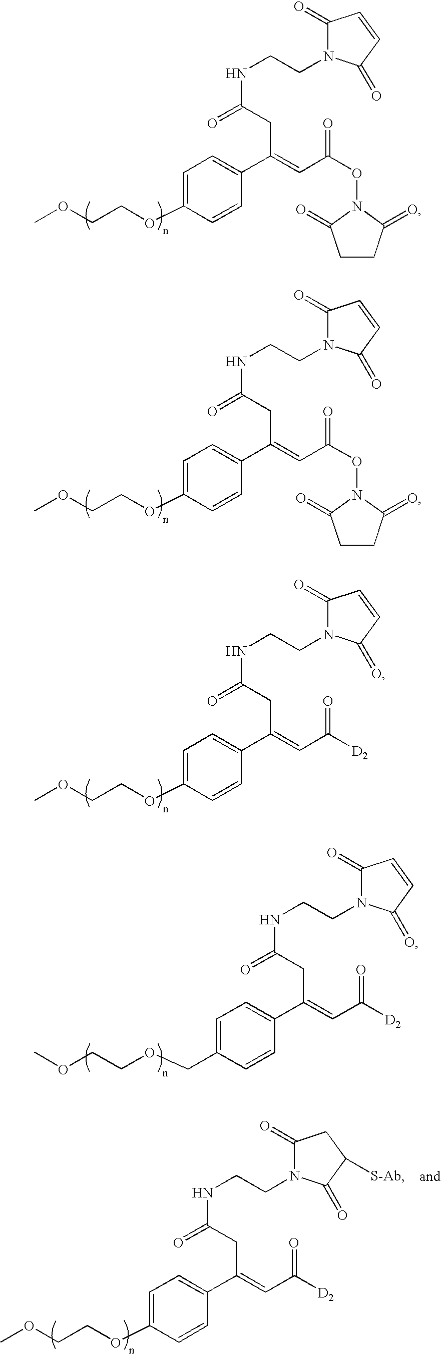

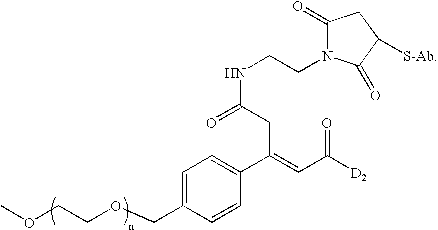

wherein

(n) is an integer from about 10 to about 2300;

D₂ is an amine containing moiety; and

Ab is an antibody.

For purposes of the present invention it will be understood that the “S-Ab” represents antibodies, such as monoclonal antibodies, single chain antibodies, and active fragments thereof.

### G. Methods of Treatment

Another aspect of the present invention provides methods of treatment for various medical conditions in mammals. The methods include administering, to the mammal in need of such treatment, an effective amount of a compound described herein. The polymeric conjugate compounds are useful for, among other things, treating diseases which are similar to those which are treated with the parent compound, e.g. enzyme replacement therapy, neoplastic disease, reducing tumor burden, preventing metastasis of neoplasms and preventing recurrences of turn or/neoplastic growths in mammals.

The amount of the polymeric conjugate that is administered will depend upon the amount of the parent molecule included therein. Generally, the amount of polymeric conjugate used in the treatment methods is that amount which effectively achieves the desired therapeutic result in mammals. Naturally, the dosages of the various polymeric conjugate compounds will vary somewhat depending upon the parent compound, molecular weight of the polymer, rate of in vivo hydrolysis, etc. Those skilled in the art will determine the optimal dosing of the polymeric transport conjugates selected based on clinical experience and the treatment indication. Actual dosages will be apparent to the artisan without undue experimentation.

The compounds of the present invention can be included in one or more suitable pharmaceutical compositions for administration to mammals. The pharmaceutical compositions may be in the form of a solution, suspension, tablet, capsule or the like, prepared according to methods well known in the art. It is also contemplated that administration of such compositions may be by the oral and/or parenteral routes depending upon the needs of the artisan. A solution and/or suspension of the composition may be utilized, for example, as a carrier vehicle for injection or infiltration of the composition by any art known methods, e.g., by intravenous, intramuscular, intraperitoneal, subcutaneous injection and the like. Such administration may also be by infusion into a body space or cavity, as well as by inhalation and/or intranasal routes. In preferred aspects of the invention, however, the polymeric conjugates are parenterally administered to mammals in need thereof.

### EXAMPLES

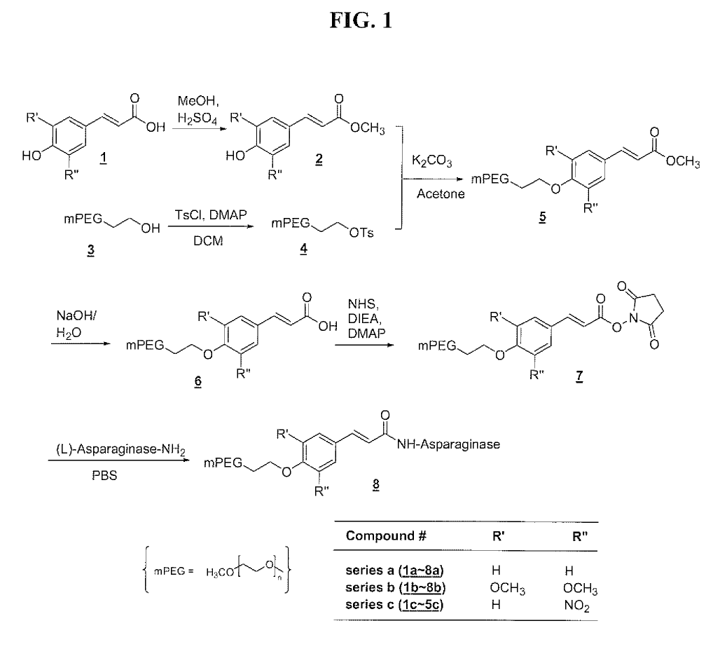

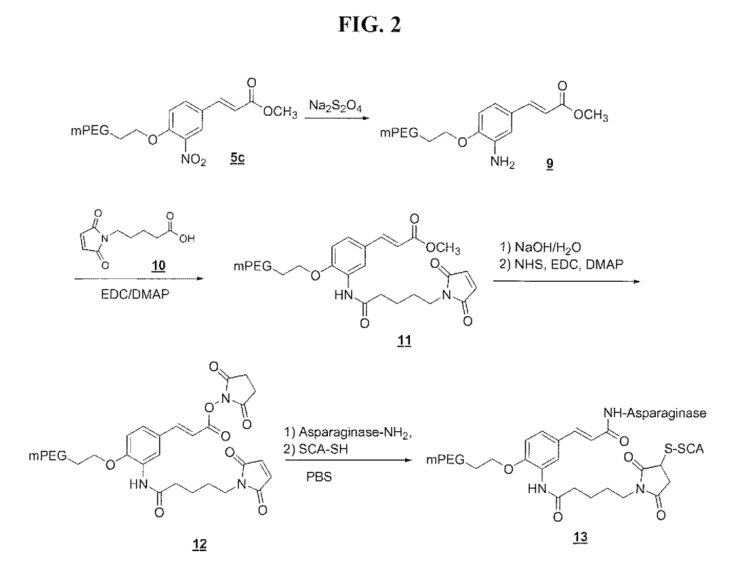

The following examples serve to provide further appreciation of the invention but are not meant in any way to restrict the scope of the invention. The bold-faced numbers recited in the Examples correspond to those shown in FIGS. 1 and 2. Abbreviations are used throughout the examples such as, DCM (dichloromethane), DIEA (diisopropylethylamine), DMAP (4-dimethylaminopyridine), DMF (N N′-dimethylformamide), DSC (disuccinimidyl carbonate), EDC (1-(3-dimethylaminopropyl)-3-ethyl carbodiimide), IPA (isopropanol), NHS(N-hydroxysuccinimide), PEG (polyethylene glycol), SCA-SH (single-chain antibody), and TEA (triethylamine).

### General

All reactions were conducted under an atmosphere of dry nitrogen. Commercial regents and anhydrous solvents were used without further purification. NMR spectra were recorded at a Varian Mercury 300 MHz NMR spectrometer using deuterated solvent indicated. Chemical shifts (6) are reported in parts per million (ppm) downfield from tetramethylsilane (TMS) and coupling constants (J values) are given in hertz (Hz).

### Example 1

### 4-Hydroxycinnamic Acid Methyl Ester (Compound 2a)

A solution of 4-hydroxycinnamic acid (compound 1a, 20.0 g, 0.12 mol) and 5 drops of concentrated sulfuric acid in 500 mL of reagent grade methanol was stirred for four days at 55° C., while monitored by TLC. The solvent was then removed from the reaction mixture on the rotovap and the solid residue was recrystallized from a mixture of 200 mL of ethanol and 200 mL of water to give 13.3 g of product in 61% yield: ¹³C NMR (75.4 MHz, CDCl₃) δ 168.18, 157.88, 144.85, 129.95, 126.91, 115.86, 114.87, 51.81.

### Example 2

### MPEG^{5K} Tosylate (Compound 4)

A solution of toluenesulfonyl chloride (21.5 g, 113 mmol) in 50 m-1 L of DCM was added to a solution of mPEG^{5K}-OH (compound 3, 113.0 g, 22.6 mmol) and of DMAP (14.1 g, 116 mmol) in 700 mL of DCM at room temperature over a period of 5 hours. This reaction mixture was then diluted with 500 mL of DCM and washed with 0.1N HCl twice. The DCM layer was dried over anhydrous sodium sulfate and filtered. The solvent was partially removed on the rotovap and the crude product was precipitated by adding ether, collected by vacuum filtration, and washed with ether. This crude product was recrystallized from 12% DMF/IPA (v/v) to give 105.0 g of product in 90% yield: ¹³C NMR (75.4 MHz, CDCl₃) δ 144.18, 132.50, 129.31, 127.42, 71.49-68.22 (PEG), 58.36, 21.32.

### Example 3

### mPEG^{5K} Cinnamic Acid Methyl Ester (Compound 5)

A mixture of mPEG^{5K} tosylate (compound 4, 40.0 g, 7.76 mmol), 4-hydroxycinnamic acid methyl ester (compound 3, 13.0 g, 73.0 mol), and anhydrous potassium carbonate (10.1 g, 73.4 mmol) in 400 mL of reagent grade acetone was refluxed overnight, followed by removal of the solvent from the reaction mixture on the rotovap. The solid residue was dissolved in DCM and washed with 0.2N HCl twice. The DCM layer was dried over anhydrous sodium sulfate and filtered. The solvent was partially removed on the rotovap and the crude product was precipitated by adding ether, collected by vacuum filtration, and washed with ether. This crude product was recrystallized from 12% DMF/IPA (v/v) to give 38.2 g of product in 95% yield: ¹³C NMR (75.4 MHz, CDCl₃) δ 167.18, 160.16, 144.04, 129.28, 126.79, 114.92, 114.58, 71.63-67.25 (PEG), 58.74, 51.30.

### Example 4

### mPEG^{5K} Cinnamic Acid (Compound 6a)

To a solution of mPEG^{5K} cinnamic acid methyl ester (compound 5a, 37.6 g, 7.26 mmol) in 300 mL of water was added a solution of sodium hydroxide (0.58 g, 14.5 mmol) in 80 mL of water. This mixture was stirred overnight at room temperature, followed by acidification of the reaction mixture with HCl, and extraction with DCM. The combined DCM layers were dried over anhydrous sodium sulfate and filtered. The solvent was then partially removed on the rotovap and the product was precipitated with ether, and collected and washed with ether to give 36.4 g of product in 97% yield: ¹³C NMR (75.4 MHz, CDCl₃) δ 168.22, 160.08, 144.42, 129.25, 126.75, 115.11, 114.50, 71.54-67.16 (PEG), 58.65.

### Example 5

### mPEG^{5K} Cinnamic Acid NHS Ester (Compound 7a)

A solution of mPEG^{5K} cinnamic acid (compound 6, 8.9 g, 1.72 mmol), NHS (0.80 g, 6.9 mmol), DIEA (1.3 g, 10.3 mmol), and DMAP (50 mg, 0.4 mmol) in 75 mL of anhydrous DCM was cooled to 0° C. in an ice bath, followed by addition of EDC HCl (1.66 g, 8.6 mmol). This mixture was allowed to warm to room temperature overnight. The solvent was partially removed on the rotovap and the product was precipitated by adding ether, collected by vacuum filtration, and washed with ether. This crude product was recrystallized from 12% DMF/IPA (v/v) to give 8.6 g of product in 96% yield. % conjugation with benzyl amine at 25° C.: T=0, 93%; T=4 weeks, 94%: ¹³C NMR (75.4 MHz, CDCl₃) δ 169.06, 161.90, 161.27, 149.31, 130.20, 126.01, 114.84, 108.38, 71.67-67.38 (PEG), 58.81, 25.47.

### Example 6

### Compound 7b

Compound 7b is prepared following the similar sequence of reactions as described in Examples 1 and 3 starting from 4-hydroxy-3,5-dimethoxycinnamic acid (compound 1b).

### Example 7

### Conjugation with (L)-Asparaginase (Compound 8a)

PEG linker (compound 7a, 0.084 mmol) is added to a solution of native (L)-asparaginase (0.00027 mmol) in 3 mL of sodium phosphate buffer (0.1 M, pH 7.8) with gentle stirring. The solution is stirred at 30° C. for 30 minutes. A GPC column (Zorbax GF-450) is used to monitor PEG conjugation. At the end of the reaction (as evidenced by the absence of native enzyme), the mixture is diluted with 12 mL of formulation buffer (0.05 M sodium phosphate, 0.85% sodium chloride, pH 7.3) and diafiltered with a Centriprep concentrator (Amicon) to remove the unreacted PEG. Dialfiltration is continued as needed at 4° C. until no more free PEG was detected by mixing equal amount of filtrate and 0.1% PMA (polymethacrylic acid in 0.1 M HCl).

### Example 8

### Compound 13

Compound 5c is prepared from 4-hydroxy-3-nitrocinnamic acid (prepared by using the procedures described in Synth. Comm. (2004) 34 (18) 3317-3324, the contents of which are incorporated herein by reference) by using the sequence of reactions similar to as described in Examples 1 and 3, and reduced using sodium thiosulfite to obtain compound 9. The amine is conjugated with 5-maleimidylpentanoic acid (compound 10) in the presence of EDC and DMAP in DCM to give compound 11. The methyl ester of compound 11 is hydrolyzed using aqueous NaOH solution and the resulting carboxylic acid is coupled with NHS in the presence of EDC and DMAP to give compound 12. Conjugation of compound 12 with asparaginase, followed by conjugation with antibody (SCA-SH) in buffered aqueous solution gives compound 13.

### Example 9

### Stability of Polymeric Delivery System

The polymeric delivery systems described herein include a compound of interest such as protein. Thus, the stability of the polymeric systems prior to conjugating to the compound of interest is important. The stability of the polymeric systems can be shown by percent (%) of conjugation to the compound.

An activated form of the polymeric systems, i.e. compound 7a containing NHS was stored at 4° C. or 25° C. After being stored for periods of 0, 1, 2, 3 and 4 weeks, the reactivity of compound 7a with a compound containing a reactive amine group, i.e. benzyl amine, was measured. Compound 7a was dissolved in DCM or chloroform followed by reacting with benzyl amine for 30 minutes at room temperature. The reaction solution was titrated with an acid to determine the amount of remaining benzyl amine and thereby calculate the amount of compound 7a conjugated. The percent (%) of conjugation is shown in Table 1.

TABLE 1
% Conjugation

| Time | % Conjugation at 4° C. | % Conjugation at 25° C. |
|---|---|---|
| 0 | 93 | 93 |
| 1 week | 92 | 94 |
| 2 week | 94 | 95 |
| 3 week | 94 | 94 |
| 4 week | 94 | 94 |

The results showed that almost same amount of compound 7a conjugated to the amine-containing compound after being stored for four weeks at 4° C. or at 25° C. The data indicates that the activated forms of the polymeric systems described herein are stable at both temperatures. This property of the polymeric systems allows large-scale production and long term storage.

We claim:

1. A compound of Formula (I): 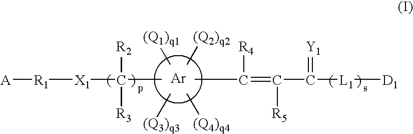
wherein:
A is a capping group or 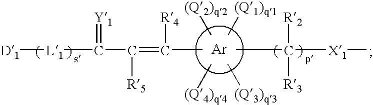
R₁ is a substantially non-antigenic water-soluble polymer;
X₁ and X′₁ are independently O, S, SO, SO₂, NR₆ or a bond;
Ar and Ar′ are independently an aryl or heteroaryl moiety;
Y₁ and Y′₁ are independently O, S, or NR₆;
L₁ and L′₁ are independently selected bifunctional linkers;
D₁ and D′₁ are independently selected from the group consisting of hydrogen, OH, leaving groups, functional groups, targeting groups, diagnostic agents and biologically active moieties;
(p) and (p′) are independently zero or a positive integer;
(q₁), (q′₁), (q₂), (q′₂), (q₃), (q′₃), (q₄) and (q′₄) are independently zero or one;
(s) and (s′) are independently zero or a positive integer:
R₂, R′₂, R₃, R′₃ and R₆ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆,alkyl, C₂₋₆ alkenyl, C₂₋₆ alkynyl, C₃₋₁₉ branched alkyl, C₃₋₈ cycloalkyl, C₁₋₆ substituted alkyl C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₁₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl substituted arylcarbonyl, C₂₋₆ substituted alkanoyloxy, substituted aryloxycarbonyl, C₂₋₆ substituted alkanoyloxy, substituted and arylcarbonyloxy, and
R₄, R′₄, R₅, R′₅, Q₁₋₄ and Q′₁₋₄ are independently selected from the same group as that which defines R₂ or 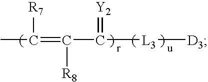
wherein
R₇ and R₈ are independently selected from the same group as that which defines R₂;
Y₂ is O, S or NR₆;
L₃ is a bifunctional linker;
(r) is zero or one;
(u) is zero or a positive integer; and
D₃ is selected from the group consisting of hydrogen, OH, leaving groups, functional groups, targeting groups and biologically active moieties;
provided that (r) is not zero when (u) is zero.

2. The compound of claim 1, wherein X₁ and X′₁ are independently O, S, SO or SO₂ when (p) is zero.

3. The compound of claim 1, wherein the sum of (q₁)+(q₂)+(q₃)+(q₄) is not zero, and at least one of R₄, R′₄, R₅, R′₅, Q₁₋₄ and Q′₁₋₄ is 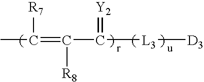
wherein D₃ is selected from the group consisting of leaving groups, functional groups, targeting groups, diagnostic agents and biologically active moieties.

4. The compound of claim 1, wherein the leaving group is selected from the group consisting of halogens, activated carbonates, carbonyl imidazole, cyclic imide thione, isocyanate, N-hydroxysuccinimidyl, para-nitrophenoxy, N-hydroxyphtalimide, N-hydroxybenzotriazolyl, imidazole, tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, imidazole, pentafluorophenoxy, 1,3,5-trichlorophenoxy, and 1,3′,5-trifluorophenoxy.

5. The compound of claim 1, wherein the functional group is selected from the group consisting of maleimidyl, vinyl, residues of sulfone, amino, carboxy, mercapto, hydrazide, and carbazate.

6. The compound of claim 1, wherein D₁, D′₁ and D₃ are independently selected from the group consisting of OH, methoxy, tert-butoxy, N-hydroxysuccinimidyl and maleimidyl.

7. The compound of claim 1, wherein the biologically active moiety is selected from the group consisting of amine containing moieties, hydroxyl containing moieties and thiol containing moieties.

8. The compound of claim 1, wherein the biologically active moiety is selected from the group consisting of pharmaceutically active compounds, enzymes, proteins, oligonucleotides, antibodies, monoclonal antibodies, single chain antibodies and peptides.

9. The compound of claim 1, wherein L₁, L′₁ and L₃ are independently selected from the group consisting of: —[C(═O)]ᵥ(CR₂₂R₂₃)ₜ[C(═O)]_{v′}—, —[C(═O)]ᵥ(CR₂₂R₂₃)ₜ—O[C(═O)]_{v′}—, —[C(═O)]ᵥ(CR₂₂R₂₃)ₜ—NR₂₆[C(═O)]_{v′}—,
—[C(—O)]ᵥO(CR₂₂R₂₃)ₜ[C(═O)]_{v′}—,
—[C(═O)]ᵥO(CR₂₂R₂₃)ₜO[C(O)]_{v′}—,
—[C(═O)]ᵥO(CR₂₂R₂₃)ₜNR₂₆[C(═O)]_{v′}—,
—[C(═O)]ᵥNR₂₁(CR₂₂R₂₃)ₜ[C(═O)]_{v′}—,
—[C(O)]ᵥNR₂₁(CR₂₂R₂₃)ₜO[C(═O)]_{v′}—,
—[C(═O)]ᵥNR₂₁(CR₂₂R₂₃)ₜNR₂₆[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥ(CR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥO(CR₂₂R₂₃)ₜO—(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁(CR₂₂R₂₃)ₜO—(CR₂₈R₂₉)_{t′}[C(═O)]_{v′}—,
—[C(═O)]ᵥNR₂₁(R₂₂R₂₃)ₜNR₂₆—(CR₂₈R₂₉)_{t′}[C(═O)]_{v′}—,
—[C(═O)]ᵥNR₂₁(CR₂₂R₂₃)ₜS—(CR₂₈R₂₉)_{t′}[C(═O)]_{v′}—,
—[C(═O)]ᵥ(CR₂₂R₂₃CR₂₈R₂₉O)ₜNR₂₆[C(═O)]_{v′}—,
—[C(═O)]ᵥ(CR₂₂R₂₃CR₂₈R₂₉)ₜ[C(═O)]_{v′}—,
—[C(═O)]ᵥO(CR₂₂CR₂₃CR₂₈R₂₉O))ₜNR₂₆[C(═O)]_{v′}—,
—[C(═O)]ᵥO(CR₂₂R₂₃CR₂₈R₂₉O)ₜ[C(—O)]_{v′}—,
—F[C(═O)]ᵥNR₂₁(CR₂₂R₂₃CR₂₈R₂₉O)ₜNR₂₆[C(═O)]_{v′}—,
—[C(═O)]ᵥNR₂₁(CR₂₂R₂₃CR₂₈R₂₉O)ₜ[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁(CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜ(CR₂₄R₂₅)_{t′}O[C(—O)]_{v′}—,
—[C(═O)]ᵥ(CR₂₂R₂₃)ₜ(CR₂₄R₂₅R₂₈R₂₉O)_{t′}[C(═O)]_{v′}—,
—[C(═O)]ᵥ(CR₂₂R₂₃)ₜ(CR₂₄R₂₅CR₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(—O)]_{v′}—,
—[C(═O)]ᵥO(CR₂₂R₂₃)ₜ(CR₂₄R₂₅CR₂₈R₂₉O)_{t′}[C(═O)]_{v′}—,
—[C(═O)]ᵥO(CR₂₂R₂₃)ₜ(CR₂₄CR₂₅CR₂₈R₂₉O)_{t′}—NR₂₆[C(═O)]_{v′}—,
—[C(═O)]ᵥNR₂₁(CR₂₂R₂₃CR₂₈R₂₉O)ₜ(CR₂₄R₂₅)_{t′}O[C(═O)]_{v′}—,
—[C(—O)]ᵥNR₂₁(CR₂₂R₂₃)ₜ(CR₂₄R₂₅CR₂₈R₂₉O)_{t′}[C(═O)]_{v′}—,
—[C(═O)]ᵥNR₂₁(CR₂₂R₂₃)ₜ(CR₂₄R₂₅CR₂₈R₂₉O)_{t′}NR₂₆[C(═O)]_{v′}—, 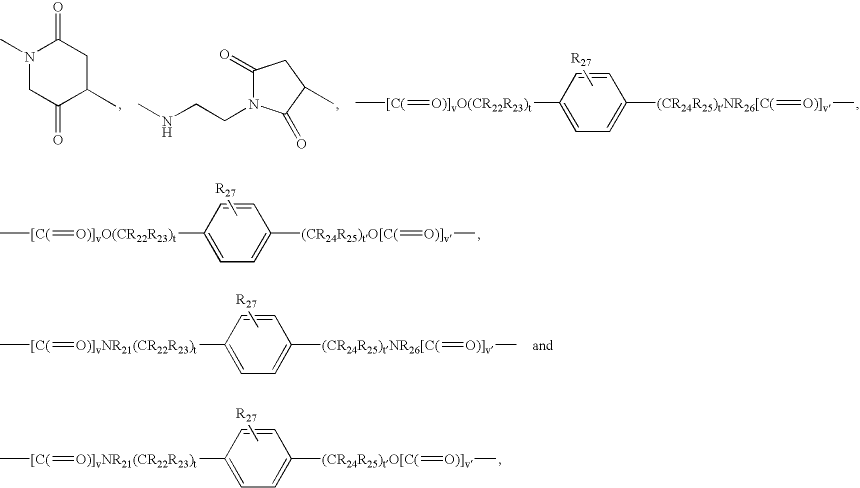
wherein:
R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t) and (t′) are independently zero or a positive integer; and
(v) and (v′) are independently zero or 1.

10. The compound of claim 1, wherein L₁, L′₁ and L₃ are independently selected from the group consisting of:
—[C(═O)]ᵣ₁NH(CH₂)₂CH═N—NHC(═O)—(CH₂)₂—,
—[C(═O)]ᵣ₁NH(CH₂)₂(CH₂CH₂O)₂(CH₂)₂NH[C(═O)]_{r1′}—,
—[C(═O)]ᵣ₁NH(CH₂CH₂)(CH₂CH₂O)₂NH[C(═O)]_{r1′}—,
—[C(═O)]ᵣ₁NH(CH₂CH₂)ₛ₁NH(CH₂CH₂)_{s1′}[C(═O)]_{r1′}—,
—[C(═O)]ᵣ₁NH(CH₂CH₂)ₛ₁(CH₂CH₂)_{s1′}[C(═O)]_{r1′}—,
—[C(═O)]ᵣ₁NH(CH₂CH₂)(CH₂CH₂O)[C(═O)]_{r1′}—,
[C(═O)]ᵣ₁NH(CH₂CH₂)ₛ₁O(CH₂CH₂)_{s1′}[C(═O)]_{r1′}—,
—[C(═O)]ᵣ₁NH(CH₂CH₂O)(CH₂)NH[C(═O)]_{r1′}—,
—[C(═O)]ᵣ₁NH(CH₂CH₂O)₂(CH₂)[C(═O)]_{r1′}—,
—[C(═O)]ᵣ₁NH(CH₂CH₂O)ₛ₁(CH₂)_{s1′}[C(═O)]_{r1′}—,
—[C(═O)]ᵣ₁NHCH₂CH₂NH[C(═O)]_{r1′}—,
—[C(═O)]ᵣ₁NH(CH₂CH₂)₂O[C(═O)]_{r1′}—,
—[C(═O)]ᵣ₁NH(CH₂CH₂O)[C(═O)]_{r1′}—,
—[C═O)]ᵣ₁NH(CH₂CH₂O)₂[C(—O)]_{r1′}—,
—[C(═O)]ᵣ₁NH(CH₂)₃[C(═O)]_{r1′}—,
—[C(═O)]ᵣ₁O(CH₂CH₂O)₂(CH₂)[C(═O)]_{r1′}—,
—[C(═O)]ᵣ₁O(CH₂)₂NH(CH₂)₂[C(═O)]_{r1′}—,
—[C(═O)]ᵣ₁O(CH₂CH₂O)₂NH[C(═O)]_{r1′}—,
—[C(═O)]ᵣ₁O(CH₂)₂O(CH₂)₂[C(═O)]_{r1′}—,
—[C(═O)]ᵣ₁O(CH₂)₂S(CH₂)₂[C(═O)]_{r1′}—,
—[C(═O)]ᵣ₁O(CH₂CH₂)NH[C(═O)]_{r1′}—,
—[C(═O)]ᵣ₁O(CH₂CH₂)O[C(═O)]_{r1′}—,
—[C(═O)]ᵣ₁O(CH₂)₃NH[C(═O)]_{r1′}—,
—[C(═O)]ᵣ₁O(CH₂)₃O[C(═O)]_{r1′}—,
—[C(═O)]ᵣ₁O(CH₂)₃O[C(═O)]_{r1′}—,
—[C(═O)]ᵣ₁CH₂NHCH₂[C(═O)]_{r1′}—,
—[C(═O)]ᵣ₁CH₂OCH₂[C(═O)]_{r1′}—,
—[C(═O)]ᵣ₁CH₂SCH₂[C(═O)]_{r1′}—,
—[C(═O)]ᵣ₁S(CH₂)₃[C(═O)]_{r1′}—,
—[C(═O)]ᵣ₁(CH₂)₃[C(═O)]_{r1′}—, 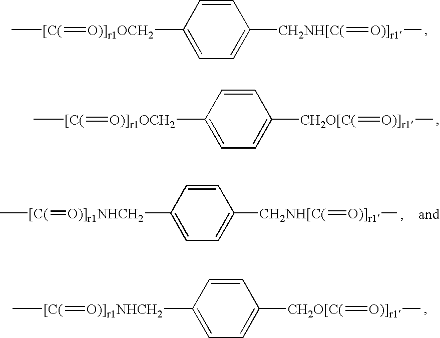
wherein
(r1) and (r1′) are independently zero or 1; and
(s1) and (s1′) are independently zero or a positive integer provided that both (r1) and (r1′) are not zero simultaneously.

11. The compound of claim 1, wherein L₁, L′₁ and L₃ are independently selected from the group consisting of amino acids, amino acid derivatives, and peptides.

12. The compound of claim 1, wherein L₃ is selected from the group consisting of:

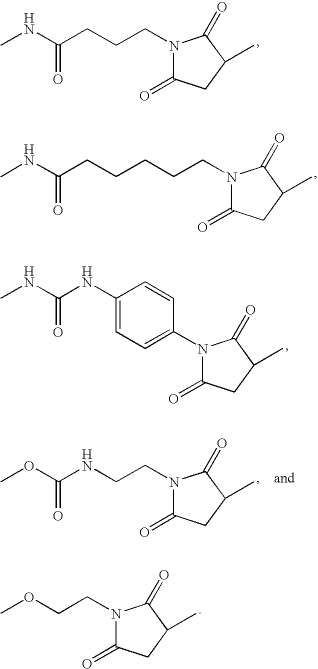

13. The compound of claim 1, wherein A is selected from the group consisting of H, NH₂, OH, CO₂H, C₁₋₆ alkoxy and C₁₋₆ alkyl.

14. The compound of claim 1, wherein A is methyl or methoxy.

15. The compound of claim 1, wherein Ar and Ar′ are independently selected from the group consisting of: 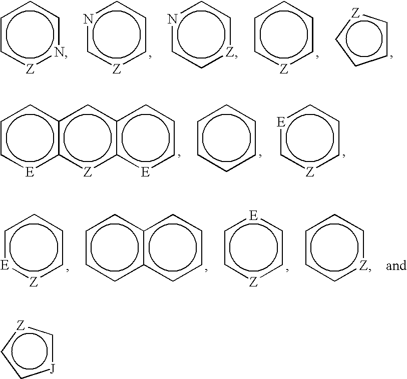
wherein
J is O, S, or NR₁₁;
E and Z are independently CR₁₂ or NR₁₃; and
R₁₁, R₁₂ and R₁₃ are independently selected from the sane group as that which defines R₂.

16. The compound of claim 1 having the formula: 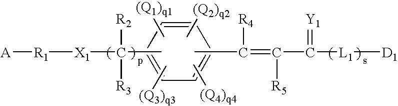
wherein A is a capping group or

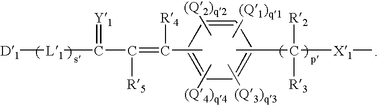

17. The compound of claim 1, having the formula:

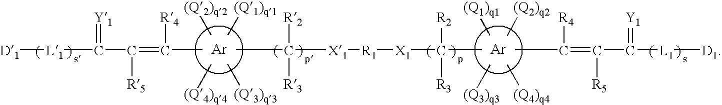

18. The compound of claim 1, wherein R₄, R′₄, R₅ and R′₅ are independently hydrogen or CH₃.

19. The compound of claim 1, wherein R₁ comprises a linear, terminally branched or multi-armed polyalkylene oxide.

20. The compound of claim 19, wherein the polyalkylene oxide is selected from the group consisting of polyethylene glycol and polypropylene glycol.

21. The compound of claim 19, wherein the polyalkylene oxide is a polyethylene glycol of the formula, —O—(CH₂CH₂O)ₙ—
wherein (n) is an integer from about 10 to about 2,300.

22. The compound of claim 19, wherein the polyalkylene oxide has an average molecular weight from about 2,000 to about 100,000 daltons.

23. The compound of claim 19, wherein the polyalkylene oxide residue has an average molecular weight of from about 5,000 to about 60,000 daltons.

24. The compound of claim 19, wherein the polyalkylene oxide has an average molecular weight from about 5,000 to about 25,000 daltons or from about 20,000 to about 45,000 daltons.

25. A compound of claim 1 selected from the group consisting of: 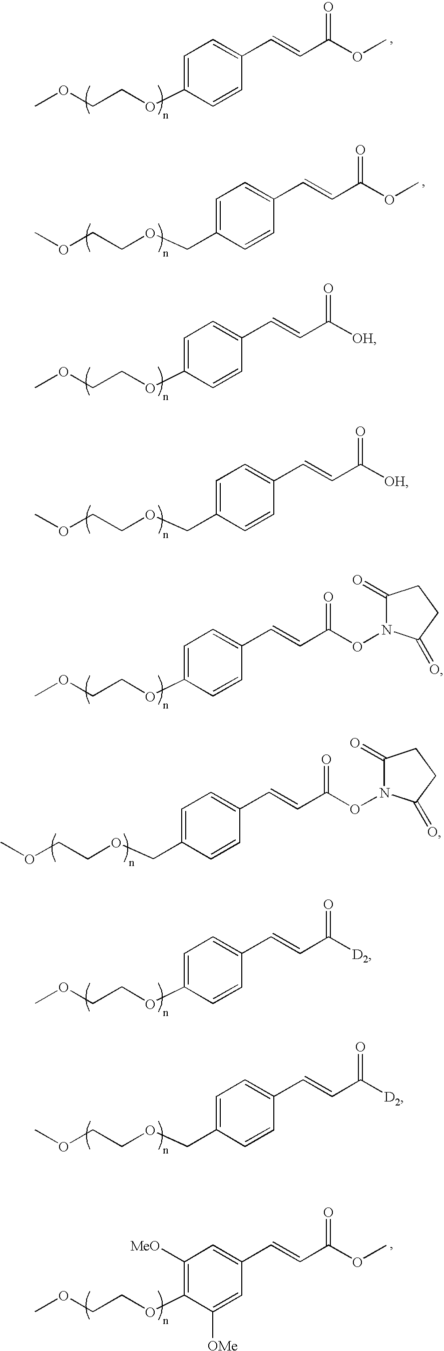 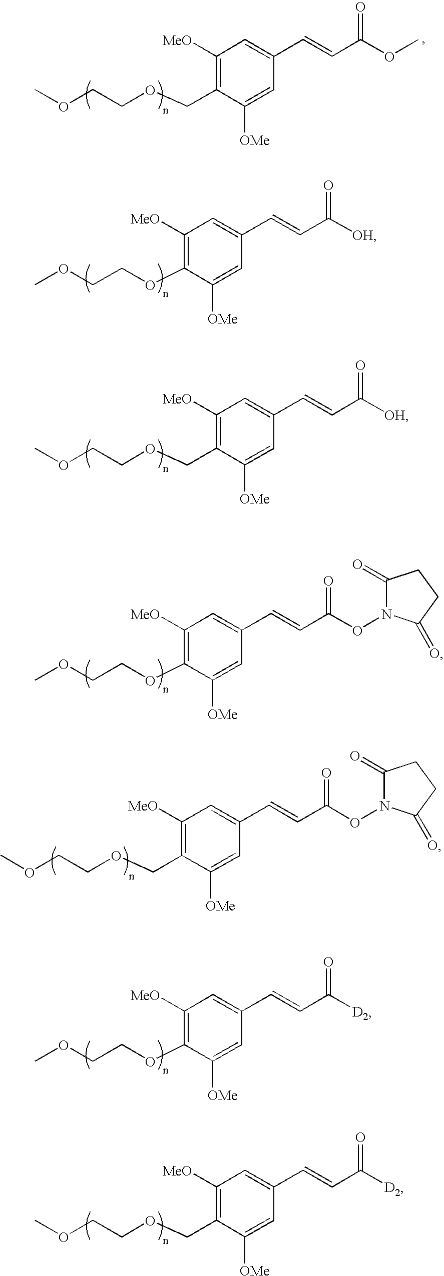 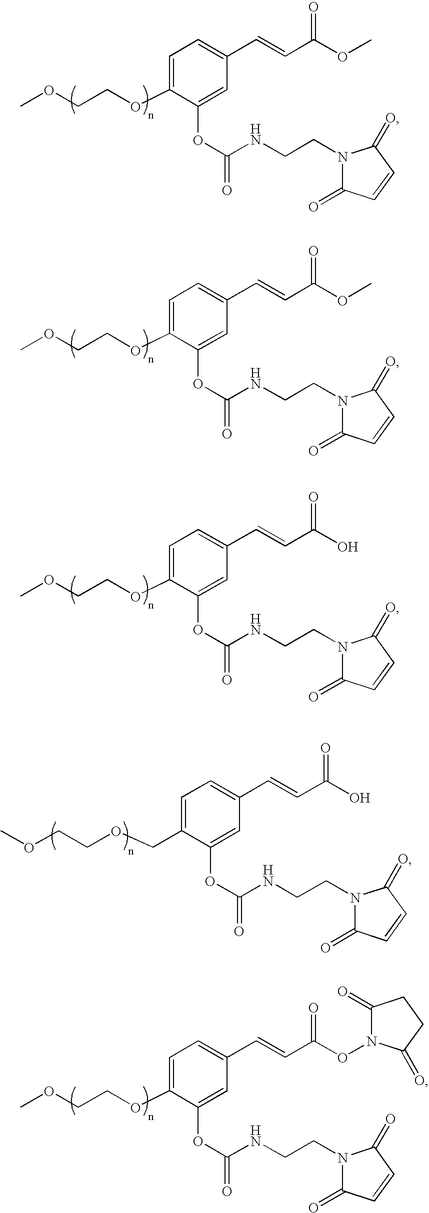 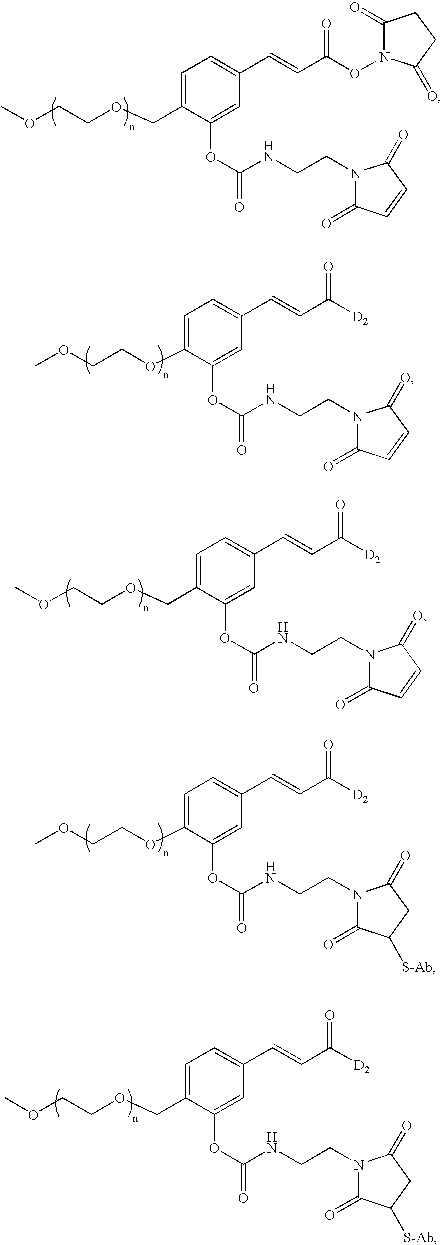 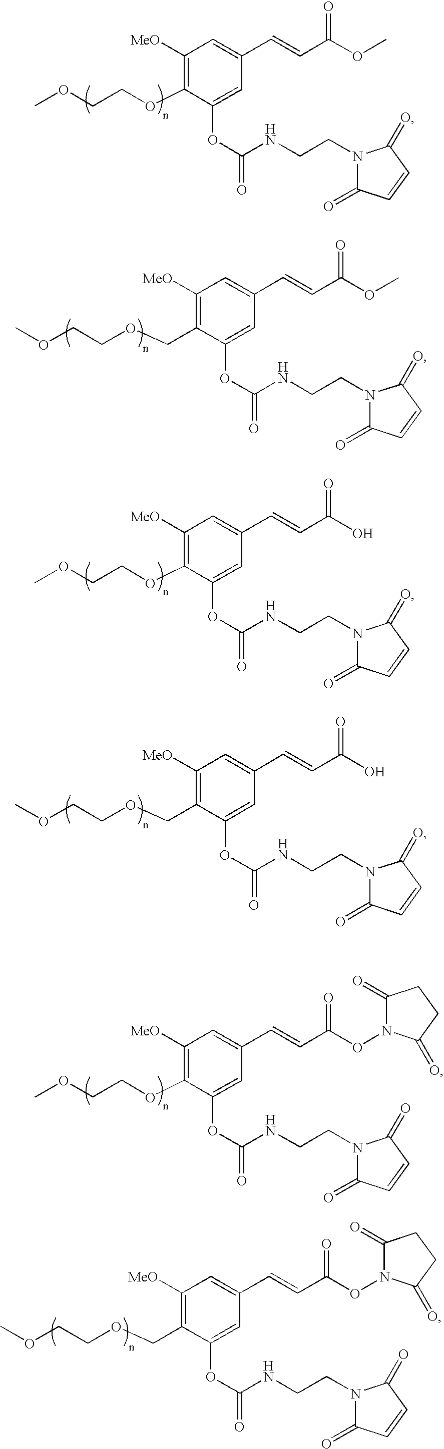 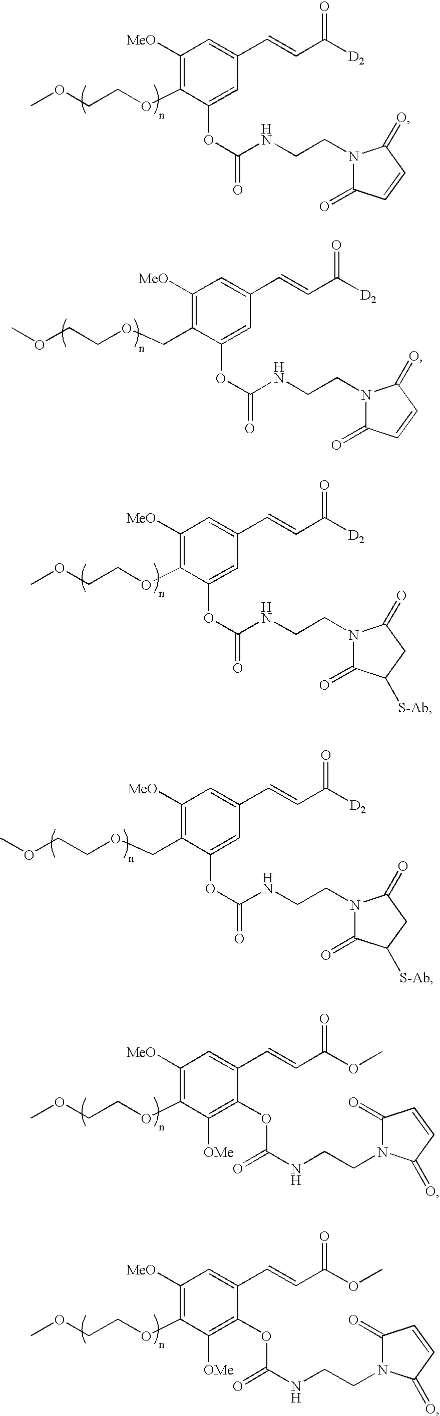 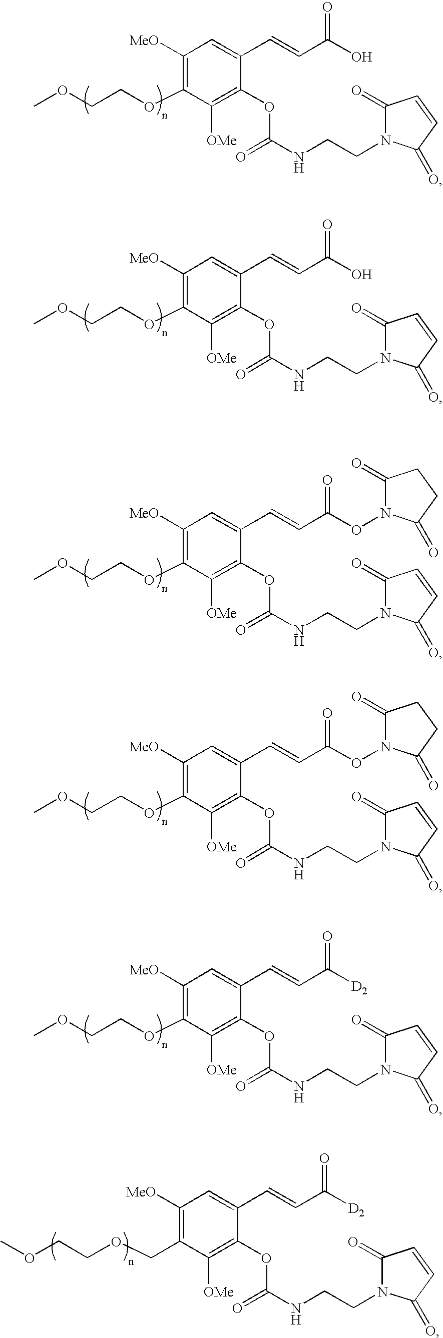 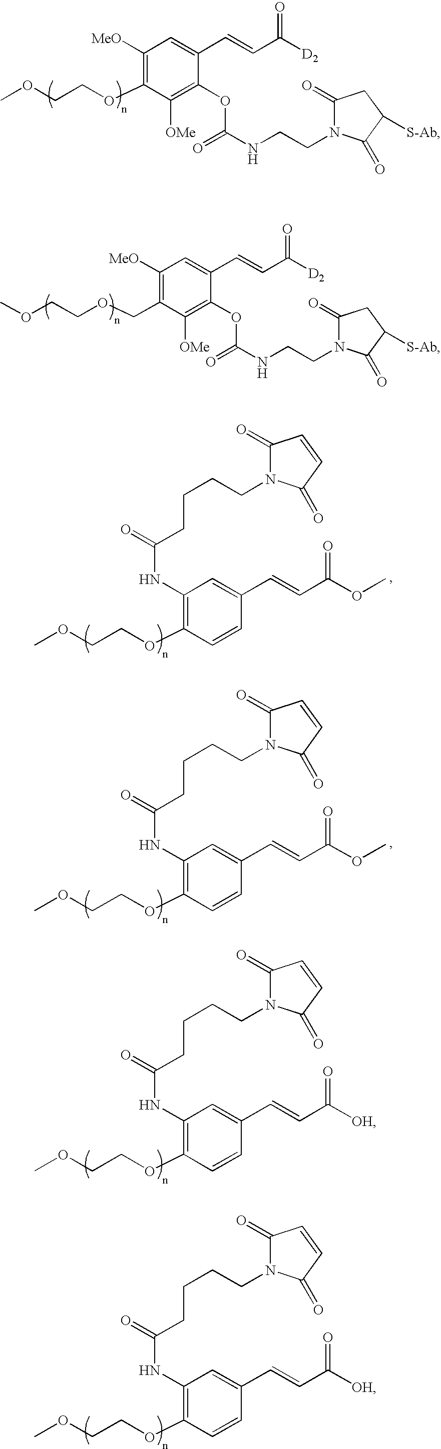 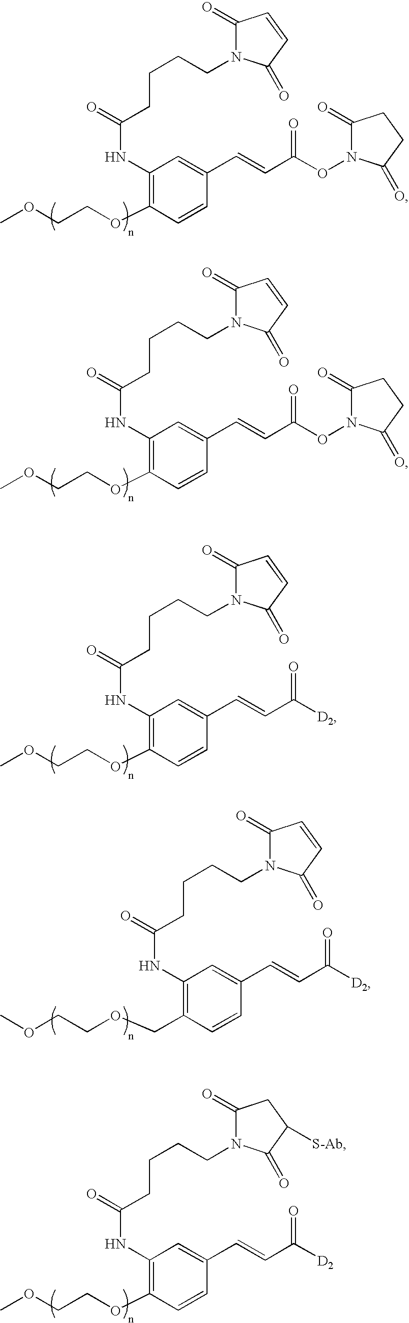 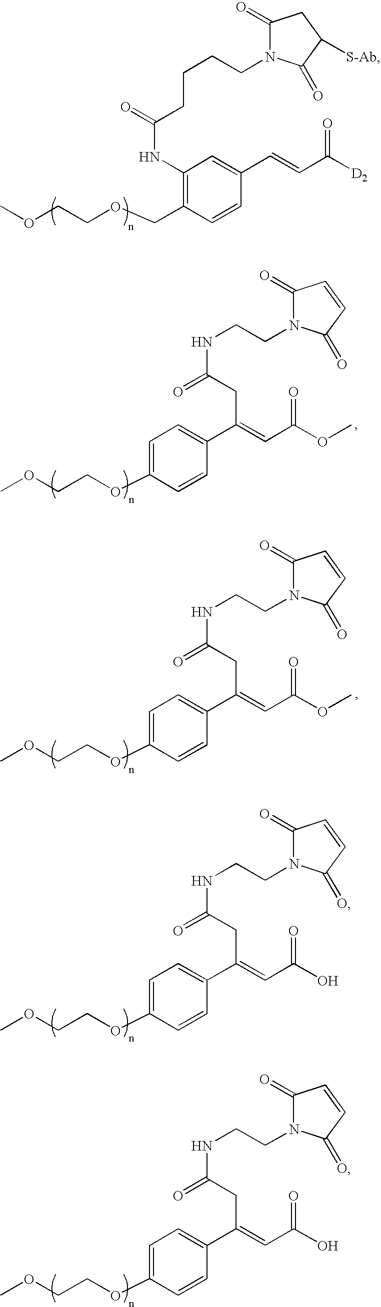 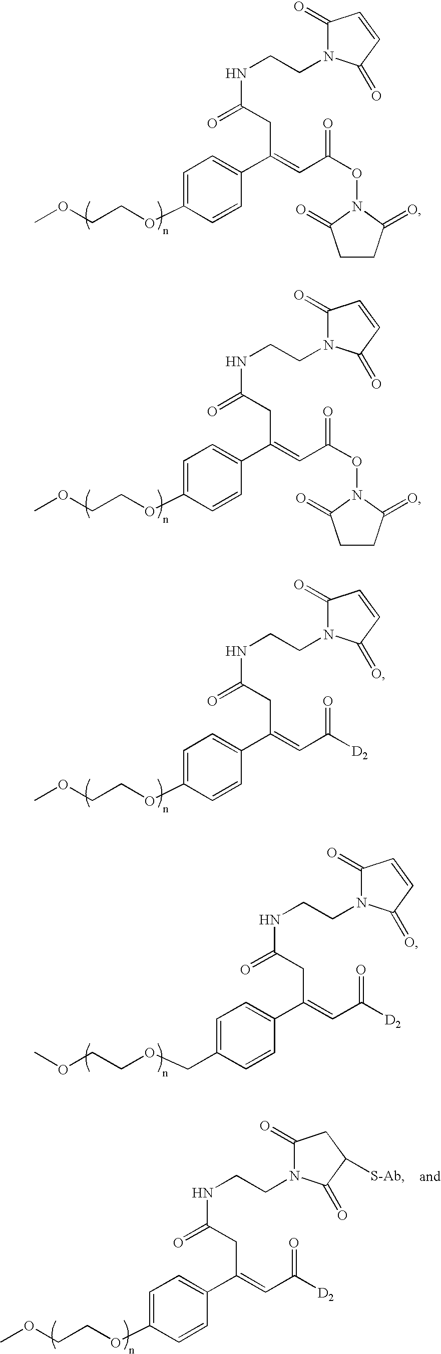 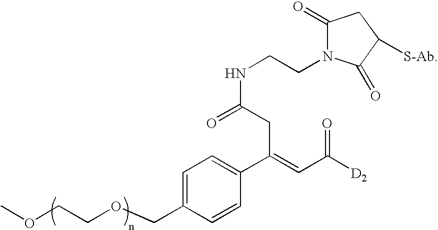
wherein
(n) is an integer from about 10 to about 2300;
D₂ is an amine containing moiety; and
Ab is an antibody.

26. The compound of claim 25 selected from the group consisting of

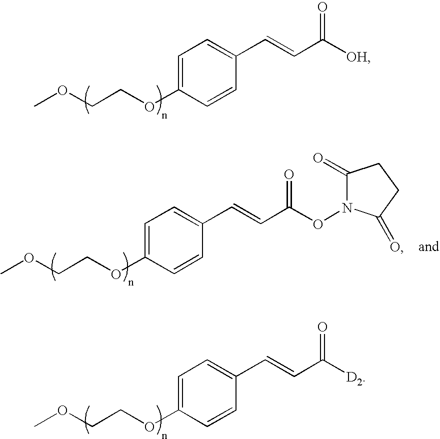

27. A method of preparing a substantially non-antigenic polymer having an aromatic allylic acid comprising:
reacting a compound of Formula (II):
A₁-R₁—X₁-M₁ (II)
with a compound of Formula (III): 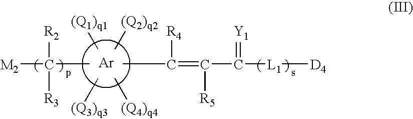
under conditions sufficient to form a compound of Formula (IV): 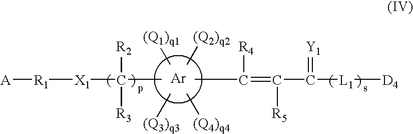
wherein:
A₁ is a capping group or M₁-X′₁—;
A is a capping group or 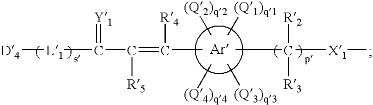
R₁ is a substantially non-antigenic water-soluble polymer;
M₁ is a leaving group;
M₂ is —OH, —SH or —NHR₄₁;
D₄ and D′₄ are independently selected from the group consisting of hydrogen, OH′, OR₄₂, functional groups and leaving groups, targeting groups, diagnostic agents and biologically active moieties;
Ar and Ar′ are independently an aryl or heteroaryl moiety;
X₁ and X′₁ are independently O, S, SO, SO₂, NR₆ or a bond;
Y₁ and Y′₁ are independently O, S, or NR₆;
L₁ and L′₁ are independently selected bifunctional linkers;
R₂, R′₂, R₃, R′₃, R₆ and R₄₁ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl. C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy;
R₄₂ is C₁₋₆ alkyl;
(p) and (p′) are independently zero or a positive integer;
(q₁), (q′₁), (q₂) (q′₂) (q₃), (q′₃), (q₄) and (q′₄) are independently zero or one;
(s) and (s′) are independently zero or a positive integer;
R₄, R′₄, R₅, R′₅, Q₁₋₄ and Q′₁₋₄ are independently selected from the same group as that which defines R₂ or 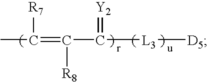
wherein,
R₇ and R₈ are independently selected from the same group as that which defines R₂;
Y₂ is O, S, NR₆;
L₃ is a bifunctional linker;
(r) is zero or one;
(u) is zero or a positive integer; and
D₅ is selected from the group consisting of hydrogen, OH, OR₄₂, functional groups and leaving groups, targeting groups, diagnostic agents and biologically active moieties; and
provided that (r) is not zero when (u) is zero.

28. A method of treating a mammal, comprising administering an effective amount of a compound of Formula (I) to a patient in need thereof, wherein at least one of D₁, D′₁, and D₃ is a biologically active moiety.
